(19) United States
(12) Patent Application Publication
(10) Pub. No.: US 2004/0156820 A1
(43) Pub. Date: Aug. 12, 2004

(54) Adjuvant combination formulations

(76) Inventor: Michael Hagen, Pittsford, NY (US)

Correspondence Address: WYETH, PATENT LAW GROUP, FIVE GIRALDA FARMS, MADISON, NJ, 07940, US

(21) Appl. No.: 10/416,262

(22) PCT Filed: Nov. 8, 2001
(86) PCT No.: PCT/US01/46943

(30) Foreign Application Priority Data
Nov. 10, 2000 (US) 60247100
Oct. 18, 2001 (US) 60330345

(51) Int. Cl. A61K 39/12; A61K 39/02; A61K 38/19; A61K 38/20

(52) U.S. Cl. 424/85.1; 424/186.1; 424/200.1; 514/62

(57) ABSTRACT

The use of an aminoalkyl glucosamine phosphate compound, or a derivative or analog thereof, in combination with a cytokine or lymphokine such as granulocyte macrophage colony stimulating factor or interleukin-12, is useful as an adjuvant combination in an antigenic composition to enhance the immune response in a vertebrate host to a selected antigen.

### FIELD OF THE INVENTION

This invention relates to the use of an aminoalkyl glucosamine phosphate compound, or a derivative or analog thereof, in combination with a cytokine or lymphokine, in particular granulocyte macrophage colony stimulating factor or interleukin-12, as an adjuvant formulation in an antigenic or immunogenic composition to enhance the immune response in a vertebrate host to a selected antigen.

### BACKGROUND OF THE INVENTION

The immune system uses a variety of mechanisms for attacking pathogens. However, not all of these mechanisms are necessarily activated after immunization. Protective immunity induced by immunization is dependent on the capacity of the immunogenic composition to elicit the appropriate immune response to resist or eliminate the pathogen. Depending on the pathogen, this may require a cell-mediated and/or humoral immune response.

The current paradigm for the role of helper T cells in the immune response is that T cells can be separated into subsets on the basis of the cytokines they produce, and that the distinct cytokine profile observed in these cells determines their function. This T cell model includes two major subsets: TH-1 cells that produce interleukin-2 (IL-2) and interferon gamma, which augment both cellular and humoral (antibody) immune responses; and TH-2 cells that produce interleukin-4, interleukin-5 and interleukin-10 (IL-4, IL-5 and IL-10, respectively), which augment humoral immune responses (Bibliography entry 1).

It is often desirable to enhance the immunogenic potency of an antigen in order to obtain a stronger immune response in the organism being immunized and to strengthen host resistance to the antigen-bearing agent. In some situations, it is desirable to shift the immune response from a predominantly humoral (TH-2) response to a more balanced cellular (TH-1) and humoral (TH-2) response.

A cellular response involves the generation of a CD8+ CTL (cytotoxic T-lymphocyte) response. Such a response is desirable for the development of immunogenic compositions against intracellular pathogens. Protection against a variety of pathogens requires strong mucosal responses, high serum titers, induction of CTL and vigorous cellular responses. These responses have not been provided by most antigen preparations, including conventional subunit immunogenic compositions. Among such pathogens is the human immunodeficiency virus (HIV).

Thus, there is a need to develop antigenic composition formulations that are able to generate both humoral and cellular immune responses in a vertebrate host.

### SUMMARY OF THE INVENTION

Accordingly, it is an object of this invention to utilize adjuvant combination formulations in antigenic compositions containing an aminoalkyl glucosamine phosphate compound (AGP), or a derivative or analog thereof, combined with a cytokine or lymphokine, in particular granulocyte-macrophage colony stimulating factor (GM-CSF) or interleukin-12 (IL-12), or an agonist or antagonist to said cytokine or lymphokine. In particular, the AGP is 2-[(R)-3-Tetradecanoyloxytetradecanoylamino]ethyl 2-Deoxy-4-O-phosphono-3-O-[(R)-3-tetradecanoyoxytetradecanoyl]-2-[(R)-3-tetradecanoyoxytetradecanoylamino]-β-D-glucopyranoside, which is also known as 529 (formerly known as RC529).

An adjuvant is a substance that enhances the immune response when administered together with an immunogen or antigen. The adjuvant formulation of this invention is administered together with a selected antigen in an antigenic or immunogenic composition. The antigenic compositions of this invention enhance the immune response in a vertebrate host to that selected antigen. The selected antigen may be a polypeptide, peptide or fragment derived (1) from a pathogenic virus, bacterium, fungus or parasite, or (2) from a cancer cell or tumor cell, or (3) from an allergen so as to interfere with the production of IgE so as to moderate allergic responses to the allergen, or (4) from amyloid precursor protein so as to prevent or treat disease characterized by amyloid deposition in a vertebrate host. In one embodiment of the invention, the selected antigen is from HIV. The selected HIV antigen may be an HIV protein, polypeptide, peptide or fragment derived from said protein. In a particular embodiment of the invention, the HIV antigen is a specific peptide. In another embodiment of the invention, the selected antigen is the β-amyloid peptide (also referred to as Aβ peptide), which is an internal, 39-43 amino acid fragment of amyloid precursor protein (APP), which is generated by processing of APP by the β and γ secretase enzymes.

The AGP can be present as an aqueous solution, or as a stabilized oil-in-water emulsion (stable emulsion or SE). In a preferred embodiment of the invention, the oil-in-water emulsion contains squalene, glycerol and phosphatidyl choline. In the SE formulation, the ACG is mixed with the cytokine or lymphokine to form the antigenic composition prior to administration. The cytokine or lymphokine is not required to enter the emulsion. In a preferred embodiment of the invention, the AGP is in the BE form. The antigenic composition may further comprise a diluent or carrier.

The invention is also directed to methods for increasing the ability of an antigenic composition containing a selected antigen (1) from a pathogenic virus, bacterium, fungus or parasite to elicit the immune response of a vertebrate host, or (2) from a cancer antigen or tumor-associated antigen from a cancer cell or tumor cell to elicit a therapeutic or prophylactic anti-cancer effect in a vertebrate host, or (3) from an allergen so as to interfere with the production of IgE so as to moderate allergic responses to the allergen, or (4) from a molecule or portion thereof which represents those produced by a host (a self molecule) in an undesired manner, amount or location so as to reduce such an undesired effect, by including an effective adjuvanting amount of a combination of a cytokine or lymphokine, in particular an AGP with GM-CSF or IL-12, or an agonist or antagonist to said cytokine or lymphokine.

The invention is further directed to methods for increasing the ability of an antigenic composition containing a selected antigen from a pathogenic virus, bacterium, fungus or parasite to elicit cytotoxic T lymphocytes in a vertebrate host by including an effective adjuvanting amount of a combination of a cytokine or lymphokine, in particular an AGP with GM-CSF or IL-12, or an agonist or antagonist to said cytokine or lymphokine.

### BRIEF DESCRIPTION OF THE FIGURES

FIG. 1 depicts the geometric mean titers of antibodies to the Aβ1-42 peptide in transgenic mice immunized as follows: Group 1—Aβ1-42 peptide plus PBS (not shown); Group 2—Aβ1-42 peptide plus MPL™ SE (squares); Group 3—Aβ1-42 peptide plus MPL™ SE and GM-CSF (triangles); Group 4—Aβ1-42 peptide plus 529 SE and GM-CSF (inverted triangles).

FIG. 2 depicts total Aβ cortical levels in transgenic mice immunized with the four Groups described for FIG. 1.

FIG. 3 depicts Aβ1-42 peptide cortical levels in transgenic mice immunized with the four Groups described for FIG. 1.

FIG. 4 depicts the frontal cortex amyloid burden in transgenic mice immunized with the four Groups described for FIG. 1.

FIG. 5 depicts the frontal cortex neuritic burden in transgenic mice immunized with the four Groups described for FIG. 1.

FIG. 6 depicts the retrosplenial cortex astrocytosis levels in transgenic mice immunized with the four Groups described for FIG. 1.

FIG. 7 depicts the HIV C4(E9V)-V3_{89.6P} peptide-specific IgG geometric mean antibody titers in serum in two groups of cynomologous macaques (four animals per group). Group 1 animals were immunized intranasally with the C4(E9V)-V3_{89.6P} peptide alone. Group 2 animals were immunized intramuscularly with the C4(E9V)-V3_{89.6P} peptide formulated with 529 SE and GM-CSF. Arrows indicate the immunizations at weeks 0, 4, 8, 18 and 23.

FIG. 8 depicts the geometric mean antibody titers in cervicovaginal lavage samples of the same animals described for FIG. 7.

FIG. 9 depicts the geometric mean antibody titers in nasal wash samples of the same animals described for FIG. 7.

### DETAILED DESCRIPTION OF THE INVENTION

Adjuvants, cytokines and lymphokines are immune modulating compounds which have the ability to enhance and steer the development and profile of immune responses against various antigens that are themselves poorly immunogenic. The appropriate selection of adjuvants, cytokines and lymphokines can induce good humoral and cellular immune responses that would not develop in the absence of adjuvant, cytokine or lymphokine. In particular, adjuvants, cytokines and lymphokines have significant effects in enhancing the immune response to subunit and peptide antigens in immunogenic compositions. Their stipulatory activity is also beneficial to the development of antigen-specific immune responses directed against protein antigens. For a variety of antigens that require strong mucosal responses, high serum titers, induction of CTL and vigorous cellular responses, adjuvant and cytokine/lymphokine combinations provide stimuli that are not provided by most antigen preparations.

Numerous studies have evaluated different adjuvant formulations in animal models, but alum (aluminum hydroxide or aluminum phosphate) is currently the only adjuvant licensed for widespread use in humans. Another adjuvant is Stimulon™ QS-21 (QS-21) (Antigenics Inc., Framingham, Mass. (2)). One group of adjuvants, stable emulsions, consisting of various water-in-oil or oil-in-water combinations, has received considerable attention for their immanopotentiating ability. These formulations generally consist of various combinations of metabolizable or inert oils, that act to stabilize and depot antigen at the site of injection. One such adjuvant is incomplete Freund's adjuvant (IFA), which includes mineral oil, water and an emulsifying agent. Complete Freund's adjuvant (CFA) is IFA plus heat-killed Mycobacteria. A particular concern in using these types of adjuvants has been injection site-associated irritation, often the result of mononuclear cell infiltrations causing granulomatous lesions. Therefore, other compounds and formulations are being investigated as potential adjuvants.

One group of such compounds are the aminoalkyl glucosamine phosphate compounds (AGPs), which are described in U.S. Pat. No. 6,113,918, for example at column 2, line 14-column 3, line 38, which is hereby incorporated by reference (3). AGPs have an aminoalkyl (aglycon) group which is glycosidically linked to a 2-deoxy-2-amino-α-D-glucopyranose (glucosamine) to form the basic structure. Further substituents include the phosphorylation of the 4 or 6 carbon on the glucosamine ring and three 3-alkanoyloxyalkanoyl residues.

One such AGP is the compound designated 529 (whose full chemical name is 2-[(R)-3-Tetradecanoyloxytetradecanoylamino]ethyl 2-Deoxy-4-O-phosphono-3-O-[(R)-3-tetradecanoyoxytetradecanoyl]-2-[(R)-3-tetradecanoyoxytetradecanoylamino]-β-D-glucopyranoside), which is produced by Corixa (Hamilton, Mont.).

Corixa also has formulated a metabolizable oil-in-water formulation which, when combined with 529, results in the formation of a stabilized emulsion designated 529 SE. The stabilized emulsion is generated through microfluidization of 529 with squalene oil, glycerol and phosphatidyl choline. The current formulation is a GMP-quality microfluidized emulsion. Emulsions containing 1% oil (although other concentrations may be used) are described in the experiments below.

529 SE resulted in no discernable gross tissue pathology when administered subcutaneously into Balb/c or Swiss-Webster mice. A stabilized emulsion containing the same components, but without 529 was also generated for comparative purposes. Specifically, subcutaneous immunization with a cysteine-deleted 39 amino acid version (−Cys) of a 40 amino acid HIV peptide T1SP10MN(A) (which lacks the cysteine residue at amino acid number 17 of the 40 amino acid peptide (+Cys)), or with Aβ1-42 (an internal, 42 amino acid fragment of APP), each formulated with the combination of adjuvants 529 SE and GM-CSF, resulted in no discernable inflammation, redness, swelling or induration.

Also within the scope of this invention are derivatives and analogs of 529 and other AGPs. Such compounds include, but are not limited to the compounds described in U.S. Pat. No. 6,113,918 (3).

The incorporation of cytokines and lymphokines into immunogenic compositions has shown promise for the expansion and enhancement of the potential of immunogenic compositions (4). The cytokine interleukin-12 (IL-12) has been demonstrated to evoke and enhance cell mediated immunity, through a shift in T helper cell subset expansion towards a Th1 cytokine profile (i.e., to IgG2 subclass in the mouse model) (5-7). In mice, recombinant murine IL-12 has been shown to enhance a Th1 dominated immune response profile (4).

IL-12 is produced by a variety of antigen-presenting cells, principally macrophages and monocytes. It is a critical element in the induction of TH1 cells from naïve T-cells. Production of IL-12 or the ability to respond to it has been shown to be critical in the development of protective TH1-like responses, for example, during parasitic infections, most notably Leishmaniasis (8), as well as enhancing the cell mediated immune response to an antigen from a pathogenic bacterium or virus (9) or from a cancer cell (10). The effects of IL-12 are mediated in large part by interferon-gamma produced by NK cells and T helper cells. Interferon-gamma is critical for the induction of IgG2a antibodies to T-dependent protein antigens (11) and IgG3 responses to T-independent antigens (12). IL-12, originally called natural killer cell stipulatory factor, is a heterodimeric cytokine (13). The expression and isolation of IL-12 protein in recombinant host cells is described in published International Patent Application WO 90/05147 (14).

Another cytokine that holds potential promise as an adjuvant is GM-CSF. GM-CSF is a particular type of colony stimulating factor (CSF). The CSFs are a family of lymphokines that induce progenitor cells found in the bone marrow to differentiate into specific types of mature blood cells. As described in U.S. Pat. No. 5,078,996 (15), which is hereby incorporated by reference, GM-CSF activates macrophages or precursor monoctyes to mediate non-specific tumoricidal activity. The nucleotide sequence encoding the human GM-CSF gene has been described (15). A plasmid containing GM-CSF cDNA has been transformed into E. coli and has been deposited with the American Type Culture Collection (ATCC), 10801 University Boulevard, Manassas, Va. 20110-2209, under Accession Number 39900. As described in U.S. Pat. No. 5,229,496 (16), which is hereby incorporated by reference, the GM-CSF gene has also been inserted into a yeast expression plasmid and deposited with the ATCC under Accession Number 53157. Furthermore, as described in U.S. Pat. No. 5,073,627 (17), which is hereby incorporated by reference, a DNA sequence encoding GM-CSF having glycosylation sites removed was deposited with the ATCC under Accession Number 67231.

GM-CSF has been shown to upregulate protein molecules on antigen presenting cells known to enhance immune responses (18), and to affect Ig secretion in sort-purified murine B cells (19). GM-CSF has also been described as an adjuvant for immunogenic compositions (20).

Other cytokines or lymphokines have been shown to have ; Ewe modulating activity, including, but not limited to, the interleukins 1-alpha, 1-beta, 2, 4, 5, 6, 7, 8, 10, 13, 14, 15, 16, 17 and 18, the interferons-alpha, beta and gamma, granulocyte colony stimulating factor, and the tumor necrosis factors alpha and beta.

Of concern related to the systemic administration of any cytokine or lymphokine are the biological consequences associated with cytokine or lymphokine activity. Additionally, cytokine or lymphokine effects related to the development of antigen-specific immune responses should be enhanced if local concentrations of cytokine or lymphokine are maintained.

The combinations of 3-O-deacylated monophosphoryl lipid A or monophosphoryl lipid A with GM-CSF or IL-12 have been evaluated; enhancement of various immune response parameters was observed (21).

The invention described herein demonstrates that, through the combination of an antigen, selected cytokine or lymphokine adjuvant, and the second adjuvant, an AGP (preferably in a stable metabolizable emulsion), the immune responses specific for the antigen are enhanced.

The antigens selected for inclusion in the antigenic compositions of this invention comprise peptides or polypeptides derived from proteins, proteins, as well as fragments of any of the following: saccharides, proteins, poly- or oligonucleotides, or other macromolecular components. As used herein, a “peptide” comprises a series of at least three amino acids and contains at least one antigenic determinant or epitope, while a “polypeptide” is a longer molecule than a peptide, but does not constitute a full-length protein. Such peptides, polypeptides or proteins may be conjugated to an unrelated protein, such as tetanus toxoid or diphtheria toxoid. As used herein, a “fragment” comprises a portion, but less than all of a saccharide, protein, poly- or oligonucleotide, or other macromolecular components. In the case of HIV, the antigenic compositions of this invention further comprise full-length HIV proteins.

The invention is first exemplified in a model system using peptide antigens derived from HIV. These peptides are described in or derived from U.S. Pat. Nos. 5,013,548 (22) and 5,019,387 (23), which are hereby incorporated by reference and are now summarized. These peptides comprise amino acid sequences which correspond to a region of the HIV envelope protein against which neutralizing antibodies and T cell responses are produced.

HIV is a human retrovirus which is the causative agent of acquired immunodeficiency syndrome (AIDS). HIV infects T lymphocytes of the immune system by attaching its external envelope glycoprotein to the CD4 (T4) molecule on the surface of T lymphocytes, thus using the CD4 (T4) molecule as a receptor to enter and infect T cells. Attempts to induce a protective immune response specific for HIV-infection through immunization have been met with very limited success. A number of approaches are currently being pursued in an attempt to determine an effective and protective strategy for the development of immunogenic compositions. These include using attenuated and recombinant bacterial vectors that express antigenic epitopes from HIV (24), recombinant adenovirus (25) or vaccinia virus vectors (26), DNA immunization (27), and synthetic peptides that contain various T and B cell epitopes of HIV (28).

The HIV external envelope glycoprotein gp120 has been shown to be capable of inducing neutralizing antibodies in man. The recombinant protein PB1, which encodes approximately one-third of the entire gp120 molecule, has been shown to include the part of the envelope protein that induces the formation of neutralizing antibodies. However, studies in chimpanzees demonstrated that neither intact gp120 or PB1 is able to induce the production of high titers of neutralizing antibodies.

Short peptides were synthesized by conventional methods which correspond to antigenic determinants of gp120 and generate an antibody response against gp120 that neutralize the virus and induce T-helper and CTL responses against the virus.

One such peptide is the C4/V3 multiepitope-containing HIV-1_{MN} peptide designated T1SP10MN(A) (+Cys), and a cysteine-deleted variant T1SP10MN(A) (−Cys). These peptides include Th, T_{CTL} and B epitopes, but do not induce antibodies which interfere with CD4 binding. Previously, it has been demonstrated that these C4/V3 HIV peptides are promising candidates for the induction of immune responses when administered with CFA, or CFA-like adjuvants (29-34). These peptides contain epitopes that have previously been shown to evoke CD4+ Th cell responses in both mice and humans, and it contains both a principal neutralizing determinant and a site which is recognized by CD8+ CTL in both Balb/c mice and humans that are HLA B7+. The 39 amino acid peptide has recently demonstrated both immunogenicity and safety in HIV-infected patients (28).

T1SP10MN(A) (+Cys) has the following sequence of 40 amino acids:

| | |
|---|---|
| Lys Gln Ile Ile Asn Met Trp Gln Glu Val Gly Lys Ala Met | (SEQ ID NO:1) |
| Tyr Ala Cys Thr Arg Pro Asn Tyr Asn Lys Arg Lys Arg Ile | |
| His Ile Gly Pro Gly Arg Ala Phe Tyr Thr Thr Lys (31). | |

T1SP10MN(A) (−Cys) has been synthesized without the cysteine at position 17 and has the following sequence of 39 amino acids:

| | |
|---|---|
| Lys Gln Ile Ile Asn Met Trp Gln Glu Val Gly Lys Ala Met | (SEQ ID NO:2) |
| Tyr Ala Thr Arg Pro Asn Tyr Asn Lys Arg Lys Arg Ile His | |
| Ile Gly Pro Gly Arg Ala Phe Tyr Thr Thr Lys. | |

This cysteine residue is located outside of the functional epitopes recognized by Th cells, CTL or B cells. Other HIV peptides from various regions of the viral genome are described in U.S. Pat. No. 5,861,243 (35), U.S. Pat. No. 5,932,218 (36), U.S. Pat. No. 5,939,074 (37), U.S. Pat. No. 5,993,819 (38), U.S. Pat. No. 6,037,135 (39), Published European Patent Application Number 671,947 (40), and U.S. Pat. No. 6,024,965 (41), which are also incorporated by reference.

A 28 amino acid peptide conjugate designated ST1/p11C is also used. The conjugate consists of a 16 amino acid SIV env-derived T-helper peptide designated ST-1, conjugated to a 12 amino acid SIV mac 251 Gag peptide (amino acids 179-190 of Gag) designated p11C (42). The p11C peptide in tetrameric form has demonstrated CTL activity in SIV mac-infected Mamu-A*01 rhesus monkeys (43). The ST1-p11C peptide conjugate has the following amino acid sequence:

| | |
|---|---|
| Arg Gln Ile Ile Asn Thr Trp His Lys Val Gly | (SEQ ID NO:3) |
| Lys Asn Val Tyr Leu Glu Gly Cys Thr Pro Tyr | |
| Asp Ile Asn Gln Met Leu; | |

A 39 amino acid peptide conjugate designated C4-V3_{89.6P} (44) is also used. The C4 region of this peptide conjugate (16 amino acids) is derived from the fourth constant region of the HIV-1 envelope protein and represents a universal T-helper epitope. The V3 portion of the peptide (23 amino acids) is derived from the third hypervariable region of the HIV-1 envelope protein and represents a critical neutralizing determinant. The C4-V3_{89.6P} conjugate has the following amino acid sequence:

| | |
|---|---|
| Lys Gln Ile Ile Asn Met Trp Gln Glu Val Gly | (SEQ ID NO:4) |
| Lys Ala Met Tyr Ala Thr Arg Pro Asn Asn Asn | |
| Thr Arg Glu Arg Leu Ser Ile Gly Pro Gly Arg | |
| Ala Phe Tyr Ala Arg Arg. | |

The HIV antigen may be a protein, polypeptide, peptide or fragment derived from said protein. The protein may be a glycoprotein such as gp41, gp120 or gp160. Alternatively, the protein may be a protein encoded by such genes as gag, pol, vif, rev, vpr, tat, nef or env. Peptides derived from such proteins will contain at least one antigenic determinant (epitope) at least six amino acids in length.

The immune response to an HIV peptide may be enhanced by covalently linking (conjugating) the peptide to a pharmaceutically acceptable carrier molecule. Examples of suitable carrier molecules include tetanus toxoid, diphtheria toxoid, keyhole limpet haemocyanin and other peptides corresponding to T cell epitopes of the HIV gp120 glycoprotein.

It is currently felt that a successful strategy for immunization against HIV will need to elicit mucosal immunity to HIV, as well as a good CTL response. In a recent urine study using the T1SP10MN(A) multi-epitope peptide, and a mucosal adjuvant, cholera toxin, it was shown that intranasal immunization induced neutralizing serum IgG1 antibodies (45). A subsequent study also using HIV-V3 loop peptides demonstrated the induction of mucosally synthesized IgA antibody and strong cell mediated responses, including peptide-specific CTL (46). The functional role of high titers of systemic and neutralizing antibodies in the prevention of, or stabilization of HIV-infected individuals is unknown, although high titers of virus-specific antibody are believed to be important in preventing viral spread.

In a preferred embodiment of the invention, a stable oil-in-water emulsion is formulated which contains 529, which is then mixed with the cytokines IL-12 or GM-CSF. The data presented below demonstrate that the combination of 529 plus GM-CSF results in high titers of HIV-neutralizing serum antibodies. The combination of 529 SE and GM-CSF induces high titers of antigen-specific IgG antibodies, including both IgG1 and IgG2a subclasses, in the vaginal vault of immunized female mice. Immunization of mice with the T1SP10MN(A) (−Cys) peptide formulated with 529 SE and GM-CSF induced a strong cellular immune response as determined by enhanced antigen specific cellular proliferation and secretion into culture of cytokines, as well as the induction of peptide-specific CTL responses. Similar results were seen when mice were immunized with the Aβ1-42 peptide from APP with 529 SE and GM-CSF.

Generally, the antigen/adjuvant formulation of 529 or 529 SE combined with GM-CSF or IL-12 and a protein or peptide of choice induces high titers of antigen-specific and virus neutralizing antibody, a significant shift in the IgG subclass ratio to a greater proportion of complement-fixing IgG antibodies (in favor of IgG2a in mice), elevated production of cytokines and cellular proliferation from mononuclear cells in response to antigen stimulation in vitro. These properties were not observed with formulations of antigen and SE in the absence of 529, either with or without GM-CSF or IL-12. The formulations of this invention also induce good cellular responses as determined through induction of CTL.

A benefit of 529 SE is that the formulation does not induce granulomatous accumulation and inflammation at the site of injection; such injection site reactions are typically induced by water-in-oil or oil-in-water adjuvant formulations.

An experiment was conducted to compare the administration of the HIV peptide T1SP10MN(A) (−Cys) with 529 SE alone, or with 529 SE plus IL-12, or 529 SE plus GM-CSF.

In this experiment (Table 1 below), Balb/c mice immunized subcutaneously with the HIV peptide T1SP10MN(A) (−Cys), formulated with 529 SE, elicited peptide-specific serum IgG titers after only two injections. The IgG1 and IgG2a subclass titers were also elicited. The inclusion of a second adjuvant, either GM-CSF or IL-12, boosted the IgG total titers, as well as the IgG1 subclass titers. The addition of IL-12 boosted the IgG2a subclass titer; the addition of GM-CSF did not boost the IgG2a subclass titer.

In another experiment, as a measure of functional cell mediated immunity, the ability of spleen cells from mice immunized with 529 SE, or 529 SE plus IL-12, or 529 SE plus GM-CSF, formulated together with the multi-epitope peptide T1SP10MN(A) (−Cys) to generate HIV_{MN}-specific CTL responses was assessed.

As shown in Table 2, spleen cells from mice immunized with any of the adjuvants demonstrated low activity toward target cells that were either unlabelled or pulse-labeled with the irrelevant IIIB CTL epitope. HIV_{MN}-specific CTL-mediated target cell lysis was markedly enhanced when 529 SE plus IL-12 was administered compared to 529 SE alone, and still further enhanced when 529 SE plus GM-CSF was administered (Table 2).

In still another experiment, rhesus monkeys were immunized with the ST1-p11C or C4-V3_{89.6P} peptides and IFA or 529 SE plus GM-CSF (groups shown in Table 15). The results of the analyses are shown in Table 16-22 and are now summarized.

The ST1-p11C peptide formulation itself seemed to be well tolerated in all the animals tested. However, significant injection site reactivities were noted with the adjuvant IFA. In addition, possible minor adverse effects of the adjuvant formulation 529 SE/GM-CSF were seen immediately after the final immunization. The ST1-p11C peptide formulation containing IFA was capable of inducing a potent p11C-specific cellular immune response in one of two Mamu-A*01 positive rhesus monkeys tested. The ST1-p11C peptide formulation containing 529 SE/GM-CSF was also capable of inducing a p11C-specific cellular immune response in one of two Mamu-A*01 positive rhesus monkeys tested.

The C4-V3_{89.6P} peptide formulation containing IFA was capable of generating peak plasma ELISA antibody titers in the range of 1:25,600-1:102,400 and serum neutralizing antibody titers against SHIV_{89.6} and SHIV_{89.6P}. The C4-V3_{89.6P} peptide formulation containing 529 SE/GM-CSF was capable of generating peak plasma ELISA antibody titers in the range of 1:6,400-1:12,800 and low level neutralizing antibody responses to SHIV_{89.6}, but not to SHIV_{89.6P}.

Given the small number of animals per group (two), it is difficult to draw concrete conclusions. However, the level of the immune response, both humoral and cellular, generated by both peptide formulations containing 529 SE/GM-CSF was qualitatively lower than the immune response seen in the animals adjuvanted with IFA. It must always be noted that IFA is not an approved component in compositions for commercial use in humans. In addition, there is some limited evidence that the functional properties and the phenotype (i.e. cytokine profiles) of the responding cells might be different depending on the adjuvant formulation used.

In yet another experiment, animals from a second primate species, cynomologous macaques, were immunized with the C4-V3_{89.6P} peptide that had been modified by changing the glutamic acid at amino acid residue 9 to valine. The resulting peptide conjugate, designated C4(E9V)-V3_{89.6P}, has the following sequence:

| | |
|---|---|
| Lys Gln Ile Ile Asn Met Trp Gln Val Val Gly | (SEQ ID NO:5) |
| Lys Ala Met Tyr Ala Thr Arg Pro Asn Asn Asn | |
| Thr Arg Glu Arg Leu Ser Ile Gly Pro Gly Arg | |
| Ala Phe Tyr Ala Arg Arg. | |

Animals were immunized with the C4(E9V)-V3_{89.6P} peptide, either without adjuvant or with the combination of 529 SE plus GM-CSF.

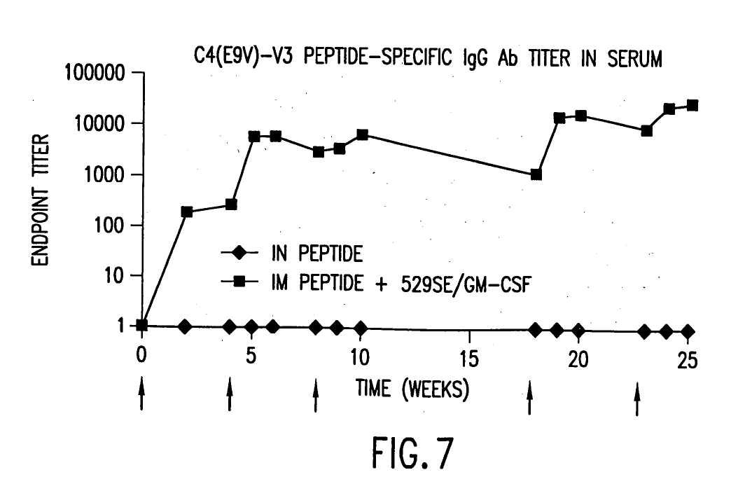

The results indicate that the C4(E9V)-V3_{89.6P} peptide when administered by intramuscular injection in combination with 529 SE/GM-CSF elicits significantly higher peptide-specific IgG titers in serum than the same amount of the C4(E9V)-V3_{89.6P} peptide administered intranasally without adjuvant (FIGS. 7-9). The results from this experiment clearly demonstrate that an HIV peptide immunogen when administered in combination with the appropriate combination of adjuvants is capable of eliciting systemic humoral immunity.

Desirable immunogenic compositions for preventing or treating disease characterized by amyloid deposition (a self molecule) in a vertebrate host, which contain the adjuvant combinations of this invention, include those containing portions of the beta amyloid precursor protein (APP). This disease is referred to variously as Alzheimer's disease, amyloidosis or amyloidogenic disease. The β-amyloid peptide (also referred to as Aβ peptide) is an internal, 39-43 amino acid fragment of APP, which is generated by processing of APP by the β and γ secretase enzymes. The Aβ1-42 peptide has the following sequence:

| | |
|---|---|
| Asp Ala Glu Phe Arg His Asp Ser Gly Tyr Glu Val His His | (SEQ ID NO:6) |
| Gln Lys Leu Val Phe Phe Ala Glu Asp Val Gly Ser Asn Lys | |
| Gly Ala Ile Ile Gly Leu Met Val Gly Gly Val Val Ile Ala. | |

In some patients, the amyloid deposit takes the form of an aggregated Aβ peptide. Surprisingly, it has now been found that administration of isolated Aβ peptide induces an immune response against the Aβ peptide component of an amyloid deposit in a vertebrate host (47). Thus, the immunogenic compositions of this invention include the adjuvant combinations of this invention plus Aβ peptide, as well as fragments, derivatives or modifications of Aβ peptide and antibodies to Aβ peptide or fragments, derivatives or modifications thereof. One such fragment of Aβ peptide is the 28 amino acid peptide having the following sequence (48):

| | |
|---|---|
| Asp Ala Glu Phe Arg His Asp Ser Gly Tyr Glu Val His His | (SEQ ID NO:7) |
| Gln Lys Leu Val Phe Phe Ala Glu Asp Val Gly Ser Asn Lys. | |

Other fragments of the Aβ peptide which are of interest include, but are not limited to, amino acids 1-10, 1-7, 1-6, 1-5, 3-7, 1-3 and 1-4, which may be administered in an unconjugated form, or conjugated to an unrelated protein.

A series of studies was conducted with the Aβ1-42 peptide and various adjuvants. A summary of the results will now be presented.

In a first experiment, Swiss-Webster mice immunized subcutaneously in the rump with the Aβ1-42 peptide generated peptide-specific antibody IgG, IgG1 and IgG2a titers, demonstrating that the Aβ1-42 peptide is a viable candidate antigen. Addition of GM-CSF to 529 SE and the Aβ1-42 peptide resulted in elevated serum antibody IgG, IgG1 and IgG2a titers compared to recipients of 529 SE and the Aβ1-42 peptide (see Tables 3-8). The serum antibodies from individual mice receiving the combination of 529 SE plus GM-CSF were elevated in more instances and were elevated more quickly than individual mice receiving 529 SE alone (data not shown). When this first experiment was repeated with older Swiss-Webster mice (6-8 months instead of less than 3 months), similar results to those in Tables 3-8 were seen (data not shown).

In a second experiment, Swiss-Webster mice were immunized subcutaneously in the rump with the Aβ1-42 peptide and 529 SE, with varying amounts of GM-CSF. IgG endpoint titers increased in a dose dependent manner as the amount of GM-CSF increased (from 0.1 to 1 to 10 μg) (Table 9). The IgG titers for all combinations of 529 SE plus GM-CSF were higher than for groups receiving another adjuvant, QS-21, alone or with GM-CSF. IgG1 subclass titers were also increased for the various 529 SE plus GM-CSF groups compared to a group which received 529 SE plus GM-CSF in a first dose and 529 SE alone in a second dose (Table 10). IgG2a subclass titers were also increased for the various 529 SE plus GM-CSF groups compared to the 529 SE alone group in a dose dependent manner (Table 11).

In a third experiment, Swiss-Webster mice were immunized subcutaneously in the rump with the Aβ1-42 peptide and 529 SE, with or without varying amounts of GM-CSF. IgG endpoint titers were increased for the various 529 SE plus GM-CSF groups (0.5 to 2 to 5 to 10 μg), although not in a dose dependent manner (Table 12). Both IgG1 and IgG2a subclass titers were also increased for the various 529 SE plus GM-CSF groups compared to the 529 SE alone group, although not in a dose dependent manner (Tables 13 [IgG1] and 14 [IgG2a]).

In a fourth experiment, transgenic mice were used which express a variant form of the β-amyloid precursor protein (APP) having a mutation at residue 717, with valine substituted by phenylalanine (49). This mutation is associated with familial Alzheimer's disease in humans. These transgenic mice (designated PDAPP mice) progressively develop many of the pathological hallmarks of Alzheimer's disease, including Aβ deposits, neuritic plaques and astrocytosis, and thus serve as an animal model for human Alzheimer's disease.

In this fourth experiment, PDAPP mice were immunized subcutaneously with the Aβ1-42 peptide with or without various adjuvants and at the dosages shown in Table A. Specifically, Group 1 mice received the Aβ1-42 peptide with MPL™ (Corixa, Hamilton, Mont.) in stable emulsion form (SE) as a positive control; Group 2 mice received the Aβ1-42 peptide with MPL™ SE plus murine GM-CSF; Group 3 mice received the Aβ1-42 peptide with 529 SE plus murine GM-CSF; Group 4 mice received PBS as a negative control. Groups 2 and 3 exhibited a more rapid increase in anti-Aβ1-42 antibody titer values as well as a higher peak titer than Groups 1 or 4. However, the titers in Groups 2 and 3 fell back to the equivalent titer of Group 1 positive controls within 2-3 months (FIG. A). Groups 1, 2 and 3 showed significant lowering of brain Aβ levels as measured by ELISA (Tables B-C and FIGS. B-C), lower amyloid burden (Table D and FIG. D) and less neuritic dystrophy (Table E and FIG. E), when compared to the Group 4 negative controls. Groups 2 and 3 had a significant reduction in astrocytosis compared to the Group 1 positive controls (FIG. F).

Thus, the adjuvanting properties of 529 SE and GM-CSF or IL-12 appear to be synergistic when formulated together.

The antigenic compositions of the present invention modulate the immune response by improving the vertebrate host's antibody response and cell-mediated immunity after administration of an antigenic composition comprising a selected antigen from a pathogenic virus, bacterium fungus or parasite, and an effective adjuvanting amount of AGP such as 529 (in an aqueous or stable emulsion form) combined with a cytokine or lymphokine, in particular GM-CSF or IL-12. Other cytokines or lymphokines have been shown to have immune modulating activity, including, but not limited to, the interleukins 1-alpha, 1-beta, 2, 4, 5, 6, 7, 8, 10, 13, 14, 15, 16, 17 and 18, the interferons-alpha, beta and gamma, granulocyte colony stimulating factor, and the tumor necrosis factors alpha and beta.

Agonists of or antagonists to certain cytokines or lymphokines are also within the scope of this invention. As used herein, the term “agonist” means a molecule that enhances the activity of, or functions in the same way as, said cytokines or lymphokines. An example of such an agonist is a mimic of said cytokines or lymphokines. As used herein, the term “antagonist” means a molecule that inhibits or prevents the activity of said cytokines or lymphokines. Examples of such antagonists are the soluble IL-4 receptor and the soluble TNF receptor.

As used herein, the term “effective adjuvanting amount” means a dose of the combination of adjuvants described herein, which is suitable to elicit an increased immune response to a selected antigen in a vertebrate host, compared to a host receiving that selected antigen in the absence of the adjuvant combination. The particular dosage will depend in part upon the age, weight and medical condition of the host, as well as on the method of administration and the antigen. In a preferred embodiment, the combination of adjuvants will utilize 529 in the range of 0.1-500 μg/dose; in a more preferred embodiment, the range is 1-100 μg/dose. Suitable doses are readily determined by persons skilled in the art. The antigenic compositions of this invention may also be mixed with immologically acceptable diluents or carriers in a conventional manner to prepare injectable liquid solutions or suspensions.

The antigenic or immunogenic compositions of this invention are administered to a human or non-human vertebrate by a variety of routes, including, but not limited to, intranasal, oral, vaginal, rectal, parenteral, intradermal, transdermal (see, e.g., International application WO 98/20734 (50) which is hereby incorporated by reference), intramuscular, intraperitoneal, subcutaneous, intravenous and intraarterial. The amount of the antigen component or components of the antigenic composition will vary depending in part upon the identity of the antigen, as well as upon the age, weight and medical condition of the host, as well as on the method of administration. Again, suitable doses are readily determined by persons skilled in the art. It is preferable, although not required, that the antigen and the combination of adjuvants be administered at the same time. The number of doses and the dosage regimen for the antigenic composition are also readily determined by persons skilled in the art. In some instances, the adjuvant properties of the combination of adjuvants may reduce the number of doses needed or the time course of the dosage regimen.

The combinations of adjuvants of this invention are suitable for use in antigenic or immunogenic compositions containing a wide variety of antigens from a wide variety of pathogenic microorganisms, including but not limited to those from viruses, bacteria, fungi or parasitic microorganisms which infect humans and non-human vertebrates, or from a cancer cell or tumor cell. The antigen may comprise peptides or polypeptides derived from proteins, as well as fragments of any of the following: saccharides, proteins, poly- or oligonucleotides, cancer or tumor cells, allergens, self molecules (such as amyloid precursor protein), or other macromolecular components. In some instances, more than one antigen is included in the antigenic composition.

Desirable viral immunogenic compositions containing the adjuvant combinations of this invention include those directed to the prevention and/or treatment of disease caused by, without limitation, Human immunodeficiency virus, Simian immunodeficiency virus, Respiratory syncytial virus, Parainfluenza virus types 1-3, Influenza virus, Herpes simplex virus, Human cytomegalovirus, Hepatitis A virus, Hepatitis B virus, Hepatitis C virus, Human papillomavirus, poliovirus, rotavirus, caliciviruses, Measles virus, Mumps virus, Rubella virus, adenovirus, rabies virus, canine distemper virus, rinderpest virus, Human metapneumovirus, avian pneumovirus (formerly turkey rhinotracheitis virus), Hendra virus, Nipah virus, coronavirus, parvovirus, infectious rhinotracheitis viruses, feline leukemia virus, feline infectious peritonitis virus, avian infectious bursal disease virus, Newcastle disease virus, Marek's disease virus, porcine respiratory and reproductive syndrome virus, equine arteritis virus and various Encephalitis viruses.

Desirable bacterial immunogenic compositions containing the adjuvant combinations of this invention include those directed to the prevention and/or treatment of disease caused by, without limitation, Haemophilus influenzae (both typable and nontypable), Haemophilus somnus, Noraxella catarrhalis, Streptococcus pneumoniae, Streptococcus pyogenes, Streptococcus agalactiae, Streptococcus faecalis, Helicobacter pylori, Neisseria meningitidis, Neisseria gonorrhoeae, Chlamydia trachomatis, Chlamydia pneumoniae, Chlamydia psittaci, Bordetella pertussis, Alloiococcus otiditis, Salmonella typhi, Salmonella typhimurium, Salmonella choleraesuis, Escherichia coli, Shigella, Vibrio cholerae, Corynebacterium diphtheriae, Mycobacterium tuberculosis, Mycobacterium avium-Mycobacterium intracellulare complex, Proteus mirabilis, Proteus vulgaris, Staphylococcus aureus, Staphylococcus epidermidis, Clostridium tetani, Leptospira interrogans, Borrelia burgdorferi, Pasteurella haemolytica, Pasteurella multocida, Actinobacillus pleuropneumoniae and Mycoplasma gallisepticum.

Desirable immunogenic compositions against fungal pathogens containing the adjuvant combinations of this invention include those directed to the prevention and/or treatment of disease caused by, without limitation, Aspergillis, Blastoqyces, Candida, Coccidiodes, Cryptococcus and Histoplasma.

Desirable immunogenic compositions against parasites containing the adjuvant combinations of this invention include those directed to the prevention and/or treatment of disease caused by, without limitation, Leishmania major, Ascaris, Trichuria, Giardia, Schistosoma, Cryptosporidium, Trichomonas, Toxoplasma gondii and Pneumocystis carinii.

Desirable immunogenic compositions for eliciting a therapeutic or prophylactic anti-cancer effect in a vertebrate host, which contain the adjuvant combinations of this invention, include those utilizing a cancer antigen or tumor-associated antigen including, without limitation, prostate specific antigen, carcino-embryonic antigen, MUC-1, Her2, CA-125 and MAGE-3.

Desirable immunogenic compositions for moderating responses to allergens in a vertebrate host, which contain the adjuvant combinations of this invention, include those containing an allergen or fragment thereof. Examples of such allergens are described in U.S. Pat. No. 5,830,877 (51) and published International Patent Application Number WO 99/51259 (52), which are hereby incorporated by reference, and include pollen, insect venoms, animal dander, fungal spores and drugs (such as penicillin). The immunogenic compositions interfere with the production of IgE antibodies, a known cause of allergic reactions.

Desirable immunogenic compositions for moderating responses to self molecules in a vertebrate host, which contain the adjuvant combinations of this invention, include those containing a self molecule or fragment thereof. Examples of such self molecules, in addition to the Aβ1-42 peptide described above, include β-chain insulin involved in diabetes, the G17 molecule involved in gastroesophageal reflux disease, and antigens which downregulate autoimmune responses in diseases such as multiple sclerosis, lupus and rheumatoid arthritis.

In the case of HIV and SIV, the antigenic compositions comprise at least one protein, polypeptide, peptide or fragment derived from said protein. In some instances, multiple HIV or SIV proteins, polypeptides, peptides and/or fragments are included in the antigenic composition.

The adjuvant combination formulations of this invention are also suitable for inclusion as an adjuvant in polynucleotide immunogenic compositions (also known as DNA immunogenic compositions). Such immunogenic compositions may further include facilitating agents such as bupivicaine (see U.S. Pat. No. 5,593,972 (53), which is hereby incorporated by reference).

In order that this invention may be better understood, the following examples are set forth. The examples are for the purpose of illustration only and are not to be construed as limiting the scope of the invention.

### EXAMPLES

### Example 1

### Materials and Methods

The following materials and methods were utilized in the experiments reported in Examples 2-7 below.

Animals

Female Balb/c mice, aged 7-9 weeks, were purchased from Taconic Farms, Inc. (Germantown, N.Y.). Female Swiss-Webster mice, aged 7-9 weeks, were also purchased from Taconic Farms, Inc. All mice were housed in a facility approved by the American Association for Accreditation of Laboratory Animal Care. Mice were acclimatized to the housing facility for one week prior to initiation of studies.

Antigens

In the HIV experiments of Examples 2-3 below, two different synthetic peptides were used. The sequence of the multiepitope HIV-1-m peptide T1SP10MN(A) (−Cys) (also referred to herein as MN-10) is as follows:

Lys Gln Ile Ile Asn Met Trp Gln Glu Val Gly Lys Ala Met Tyr Ala Thr Arg Pro Asn Tyr Asn Lys Arg Lys Arg Ile His Ile Gly Pro Gly Arg Ala Phe Tyr Thr Thr Lys (SEQ ID NO:2). This peptide has been previously described (33,34), and contains sequences from HIV-1 gp120_{MN} that evoke CD4⁺ Th cell responses in both mice and humans, a principal neutralizing determinant, and a site recognized by CD8⁺ CTL in Balb/c mice. The peptide was provided by Dr. R. Scearce (Duke University, Durham, N.C.). For CTL analysis, an irrelevant peptide designated IIIB was used for comparison purposes. This peptide corresponded to the CTL epitope within the V3 loop of HIV-1-_{IIIB} (Arg Gly Pro Gly Arg Ala Phe Val Thr Ile (SEQ ID NO:8)), and was purchased from Genosys Biotechnologies Inc. (The Woodlands, Tex.). Peptides were solubilized in sterile water, and diluted in appropriate buffers, or cell culture medium, prior to use.

In the amyloid experiments of Examples 4-6 and 8 below, a peptide designated Aβ1-42 was used. The sequence of Aβ1-42 is as follows:

| | |
|---|---|
| Asp Ala Glu Phe Arg His Asp Ser Gly Tyr Glu Val His His | (SEQ ID NO:6) |
| Gln Lys Leu Val Phe Phe Ala Glu Asp Val Gly Ser Asn Lys | |
| Gly Ala Ile Ile Gly Leu Met Val Gly Gly Val Val Ile Ala. | |

This peptide has been previously described (47) and corresponds to an internal, 42 amino acid fragment of amyloid precursor protein. The Aβ1-42 was provided by Elan Pharmaceuticals (South San Francisco, Calif.). The peptide was solubilized in sterile water, and diluted in appropriate buffers, or cell culture medium, prior to use.

Adjuvants

All 529-containing adjuvant preparations were obtained from Corixa (Hamilton, Mont.). 529 SE was prepared as a preformulated squalene based oil-in-water (0.8-2.5% oil) emulsion, having 529 concentrations ranging from (0-50 μg/ml). Aluminum phosphate was prepared in-house. Freund's complete adjuvant (CFA) and incomplete adjuvant (IFA) were purchased from Difco Laboratories, Detroit, Mich. T1SP10MN(A) peptides and Freund's adjuvants were emulsified in a 1:1 ratio using two linked syringes. Recombinantly expressed urine IL-12 was provided by Genetics Institute (Cambridge, Mass.). Recombinant murine GM-CSF was purchased from Biosource International (Camarillo, Calif.) as a carrier-free lyophilized powder. Stimulon™ QS-21 was purchased from Antigenics Inc. (Framingham, Mass.).

Immunizations

Mice were immunized subcutaneously in the rump, in a total volume of 0.2 ml equally divided on each side of the rump. Immunizations were administered at varying time intervals, as indicated below. Antigen and cytokines were diluted in phosphate buffered saline to the appropriate concentrations and formulated with adjuvants less than 16 hours prior to immunization, under sterile conditions. Immunogenic compositions were mixed by gentle agitation, and stored at 4° C. Formulations were mixed by vortex immediately prior to immunization.

Analysis of Serum using Enzyme-linked Immunosorbent Assays

Animals were bled prior to initial immunization, and at indicated time points. Analysis of serum was as a geometric mean of individual titers. For analysis of HIV peptide-specific antibody and subclass distribution, peptide was suspended in either carbonate buffer (15 mM Na₂CO₃, 35 mM NaHCO₃, pH 9.6), or PBS, at a concentration of 1 μg/ml and plated to 96 well microtiter plates (Nunc) in a volume of 100:1. After overnight incubation at 37° C., plates were washed, and blocked (0.1% gelatin/PBS) at room temperature for 2-4 hours. ELISA plates were washed with wash buffer (PBS, 0.1% Tween™20) before addition of serially diluted serum (PBS, 0.1% gelatin, 0.05% Tween™20, 0.02% sodium azide). After a four hour incubation, wells were washed and appropriate dilutions of biotinylated anti-isotype/subclass antibodies were added for incubation at 4° C. overnight. Wells were washed and incubated with strepavidin-conjugated horseradish peroxidase. After incubation, wells were washed, and developed with ABTS. Wells were read at 405 nm. Titers were standardized using control sera.

The same protocol was followed for analysis of 41-42 peptide-specific antibody and subclass distribution, except that a concentration of 0.3 μg/ml per microtiter plate was used.

Cell Preparations

For proliferation assays and in vitro cytokine analysis, spleen cells were obtained from mice at the indicated time points. Single cell suspensions were prepared from pools of 3-5 mice. For proliferation and cytokine analysis, cells were suspended in round bottom 96 well culture plates precoated overnight with HIV peptide antigens, control proteins, or RPMI-10 only. Spleen cells were added at 5×10⁵ cells/well using culture medium having 2× supplements. Cell culture supernatants were harvested from triplicate wells for cytokine analysis three or six days after culture initiation. Immediately after supernatant harvest, cultures were pulsed with ³H-thymidine for 18-24 hours, and harvested to quantify cell proliferation.

### Example 2

### Reciprocal Anti-T1SP10MN(A) (−Cys) IgG Endpoint Total and Subclass Titers

Reciprocal endpoint IgG subclass titers were measured from pooled serum (n=5 Balb/c) five weeks after initial immunization, two weeks after secondary immunization. Mice were immunized subcutaneously in the rump with 25 μg of T1SP10MN(A) (−Cys), with a total of 0.2 ml divided equally into two 0.1 ml injections on each side, at week 0 and week 3. 529 SE was diluted to create an emulsion containing 1.25% squalene oil and 25 μg 529 per dose. SE is an oil-in-water emulsion vehicle consisting of squalene, glycerol, and an emulsifying agent. Recombinant murine IL-12 was delivered at 40 ng/mouse. Recombinant murine GM-CSF was delivered at 25 μg/mouse. The results are given in Table 1, with the geometric mean titers plus standard errors for each group.

TABLE 1
Reciprocal anti-T1SP10MN(A)(-Cys)
IgG endpoint total and subclass titers

| | μg HIV | Endpoint Titers | | |
|---|---|---|---|---|
| Adjuvants | peptide | IgG | IgG1 | IgG2a |
| 529 (25) SE | 25 | 206,301 +/− 175,149 | 37,567 +/− 31,526 | 180,671 +/− 277,211 |
| (1.25% oil) | | | | |
| 529 (25) SE | 25 | 460,516 +/− 690,712 | 74,269 +/− 169,868 | 222,446 +/− 400,716 |
| (1.25% oil) + rIL-12 | | | | |
| (.04) | | | | |
| 529 (25) SE | 25 | 1,085,658 +/− 1,064,924 | 238,379 +/− 62,199 | 117,657 +/− 25,301 |
| (1.25% oil) + GM- | | | | |
| CSF (25) | | | | |

### Example 3

### CTL Analysis in Balb/c Mice

The protocols of Example 2 were followed regarding immunization of mice. The CTL activity of spleen cells isolated from mice 14 days after secondary immunization was assessed. 529 SE was formulated with 25 μg 529 SE containing 1.25% oil, with or without 10 μg GM-CSF or 40 ng IL-12, plus 25 μg T1SP10MN(A) (−Cys).

For CTL analysis, spleen cells were removed from immunized mice 14 days after secondary immunization. A protocol previously described (39) was essentially followed. Briefly, erythrocyte-depleted spleen cells from three mice per group were pooled. Spleen effector cells (4×10⁶/ml) were restimulated in 24 well culture plates in a volume of 1.5-2 ml for seven days with 1 μg/ml of either the “MN-10” peptide, the “IIIB” 10mer CTL epitope peptide, or no HIV peptide. Both CTL epitopes were restricted to H-2De. Cultures were supplemented with 10 U/ml recombinant murine IL-2 (Biosource) for the last five days of culture. For analysis of cytotoxic activity, P815 cells were labeled with Cr⁵¹ and pulsed with 5 μg/ml peptide (IIIB or MN-10) for four hours, and added to cultured splenic effector cells. When no HIV peptide was used, that set of target cells was not pulsed. Three-fold dilutions of effector to target cell ratios (“E:T”) were used, from 30:1 through 1.1:1. Percent CTL activity was calculated as the percentage of chromium release using ((specific chromium release−spontaneous chromium release)/(maximal chromium release−spontaneous chromium release))×100. Chromium release was assessed after a six hour incubation period. The average spontaneous release of chromium was always less than 15% of maximal release. The results of data from day 28 are shown in Table 2.

TABLE 2
Effector to Target Ratios

| 529 SE+: | | | | | | | | | |
|---|---|---|---|---|---|---|---|---|---|
| E:T % | Peptide MN-10 | | | Peptide IIIB | | | No Peptide | | |
| specific | | | GM- | | | GM- | | | GM- |
| release | * | IL-12 | CSF | * | IL-12 | CSF | * | IL-12 | CSF |
| 30:1 | 31 | 53 | 71 | 8 | 11 | 19 | 4 | 8 | 8 |
| 10:1 | 19 | 41 | 70 | 5 | 8 | 21 | 2 | 5 | 9 |
| 3.3:1 | 5 | 11 | 35 | 2 | 1 | 5 | −2 | −2 | −1 |
| 1.1:1 | 2 | 4 | 14 | 1 | 0 | 2 | −3 | −2 | −3 |

* No additional adjuvant

### Example 4

### Reciprocal Anti-Aβ1-42 IgG Endpoint Total and Subclass Titers

Outbred Swiss-Webster mice were divided into groups of ten mice each. Each group received 30 μg of Aβ1-42 peptide, which corresponds to an internal 42 amino acid long region of APP. The first group did not receive an adjuvant; the second group received 50 μg of 529 SE containing 2.5% oil; the third group received 50 μg of 529 SE containing 2.5% oil plus 10 μg GM-CSF; the fourth group received 10 ng GM-CSF; the fifth group received SE containing 1.25% oil; the sixth group received SE containing 1.25% oil plus 10 μg GM-CSF; the seventh group received 50 μg QS-21. Mice were immunized subcutaneously in the rump with a total volume of 0.2 ml, divided equally into each of two sites at the base of the tail/rump. Immunizations were administered at week 0 and week 3.

Mice were bled at days 0, 20, 35 and 70. Serum was analyzed from individual mice. Reciprocal endpoint anti-Aβ1-42 peptide IgG endpoint total class and subclass titers were measured from individual sera (n=10 Swiss-Webster) at week 5 and at week 10. The IgG endpoint results are given in Tables 3 (week 5) and 4 5 (week 10). The IgG1 subclass results are given in Tables 5 (week 5; the groups receiving no adjuvant or QS-21 were not measured) and 6 (week 10). The IgG2a subclass results are given in Tables 7 (week 5; the groups receiving no adjuvant or QS-21 were not measured) and 8 (week 10).

TABLE 3
Anti-Aβ1-42 Week 5 IgG Endpoint Titers

| Adjuvants | Geometric Mean | Standard Error |
|---|---|---|
| None | 12,976 | +/−14,386 |
| 529 SE (50) | 16,204 | +/−225,221 |
| 529 SE (50) + GM- | 608,474 | +/−623,575 |
| CSF (10) | | |
| GM-CSF (10) | 214,497 | +/−609,067 |
| SE (1%) | 33,342 | +/−15,493 |
| SE (1%) + GM-CSF | 453,367 | +/−162,750 |
| (10) | | |
| QS-21 (50) | 4,076 | +/−9,036 |

TABLE 4
Anti-Aβ1-42 Week 10 IgG Endpoint Titers

| Adjuvants | Geometric Mean | Standard Error |
|---|---|---|
| None | 21,426 | +/−24,959 |
| 529 SE (50) | 86,847 | +/−187,792 |
| 529 SE (50) + GM- | 943,075 | +/−989,177 |
| CSF (10) | | |
| GM-CSF (10) | 1,049,414 | +/−390,525 |
| SE (1%) | 255,631 | +/−114,025 |
| SE (1%) + GM-CSF | 1,005,899 | +/−407,108 |
| (10) | | |
| QS-21 (50) | 47,222 | +/−159,775 |

TABLE 5
Anti-Aβ1-42 Week 5 IgG1 Endpoint Titers

| Adjuvants | Geometric Mean | Standard Error |
|---|---|---|
| 529 SE (50) | 461 | +/−627 |
| 529 SE (50) + GM- | 1,936 | +/−12,680 |
| CSF (10) | | |
| GM-CSF (10) | 8,654 | +/−10,100 |
| SE (1%) | 4,515 | +/−6,273 |
| SE (1%) + GM-CSF | 24,422 | +/−19,764 |
| (10) | | |

TABLE 6
Anti-Aβ1-42 Week 10 IgG1 Endpoint Titers

| Adjuvants | Geometric Mean | Standard Error |
|---|---|---|
| None | 2,086 | +/−2,448 |
| 529 SE (50) | 969 | +/−521 |
| 529 SE (50) + GM- | 2,076 | +/−4,901 |
| CSF (10) | | |
| GM-CSF (10) | 8,483 | +/−10,998 |
| SE (1%) | 3,623 | +/−3,456 |
| SE (1%) + GM-CSF | 12,472 | +/−11,502 |
| (10) | | |
| QS-21 (50) | 988 | +/−895 |

TABLE 7
Anti-Aβ1-42 Week 5 IgG2a Endpoint Titers

| Adjuvants | Geometric Mean | Standard Error |
|---|---|---|
| 529 SE (50) | 2,224 | +/−10,099 |
| 529 SE (50) + GM- | 94,764 | +/−849,173 |
| CSF (10) | | |
| GM-CSF (10) | 25,554 | +/−13,191 |
| SE (1%) | 1,484 | +/−2,271 |
| SE (1%) + GM-CSF | 8,405 | +/−31,303 |
| (10) | | |

TABLE 8
Anti-Aβ1-42 Week 10 IgG2a Endpoint Titers

| Adjuvants | Geometric Mean | Standard Error |
|---|---|---|
| None | 5,910 | +/−39,626 |
| 529 SE (50) | 5,944 | +/−9,100 |
| 529 SE (50) + GM- | 47,694 | +/−88,053 |
| CSF (10) | | |
| GM-CSF (10) | 64,910 | +/−54,824 |
| SE (1%) | 2,350 | +/−2,326 |
| SE (1%) + GM-CSF | 7,421 | +/−31,153 |
| (10) | | |
| QS-21 (50) | 3,544 | +/−26,332 |

### Example 5

### Reciprocal Anti-Aβ1-42 Endpoint Total and Subclass Titers with Varying Amounts of GM-CSF

Outbred Swiss-Webster mice were divided into groups of ten mice each. Each group received two immunizations of 30 μg of Aβ1-42 peptide at weeks 0 and 3. The first group received 25 μg of 529 SE plus 10 μg GM-CSF; the second group received 25 μg of 529 SE plus 1 μg GM-CSF; the third group received 25 μg of 529 SE plus 0.1 μg GM-CSF; the fourth group received 25 μg of 529 SE plus 10 μg GM-CSF in the priming dose, followed by 25 μg 529 SE only in the second dose; the fifth group received 25 μg QS-21; the sixth group received 25 μg QS-21 plus 10 μg GM-CSF. Mice were immunized subcutaneously in the rump with a total volume of 0.2 ml, divided equally into each of two sites at the base of the tail/rump.

Mice were bled at days 0, 21 and 42. Reciprocal endpoint anti-Aβ1-42 peptide IgG class and subclass titers were measured from individual serum (n=10) at week 6. The IgG endpoint results are given in Table 9. The IgG1 subclass results are given in Table 10. The IgG2a subclass results are given in Table 11.

TABLE 9
Anti-Aβ1-42 Week 6 IgG Endpoint Titers

| | | |
|---|---|---|
| Adjuvants | Geometric Mean | Standard Error |

| | | |
|---|---|---|
| 529 SE (25) + GM- | 353,660 | +/−148,940 |
| CSF (10) | | |
| 529 SE (25) + GM- | 150,935 | +/−218,332 |
| CSF (1) | | |
| 529 SE (25) + GM- | 86,145 | +/−91,724 |
| CSF (0.1) | | |
| 529 SE (25)* | 25,365 | +/−54,083 |
| QS-21 (25) | 1,866 | +/−18,430 |
| QS-21 (25) + GM- | 48,970 | +/−116,106 |
| CSF (10) | | |

*First dose 529 SE (25) + GM-CSF (10);
second dose 529 SE (25) alone

TABLE 10
Anti-Aβ1-42 Week 6 IgG1 Endpoint Titers

| Adjuvants | Geometric Mean | Standard Error |
|---|---|---|
| 529 SE (25) + GM- | 10,867 | +/−18,333 |
| CSF (10) | | |
| 529 SE (25) + GM- | 24,909 | +/−18,625 |
| CSF (1) | | |
| 529 SE (25) + GM- | 6,608 | +/−17,736 |
| CSF (0.1) | | |
| 529 SE (25)* | 4,511 | +/−8,154 |
| QS-21 (25) | 581 | +/−126 |
| QS-21 (25) + GM- | 7,618 | +/−29,145 |
| CSF (10) | | |

* First dose 529 SE (25) + GM-CSF (10);
second dose 529 SE (25) alone

TABLE 11
Anti-Aβ1-42 Week 6 IgG2a Endpoint Titers

| Adjuvants | Geometric Mean | Standard Error |
|---|---|---|
| 529 SE (25) + GM- | 243,758 | +/−354,383 |
| CSF (10) | | |
| 529 SE (25) + GM- | 116,222 | +/−143,140 |
| CSF (1) | | |
| 529 SE (25) + GM- | 98,018 | +/−391,797 |
| CSF (0.1) | | |
| 529 SE (25)* | 16,018 | +/−165,298 |
| QS-21 (25) | not done | +/−not done |
| QS-21 (25) + GM- | 30,133 | +/−134,774 |
| CSF (10) | | |

*First dose 529 SE (25) + GM-CSF (10);
second dose 529 SE (25) alone

### Example 6

### Reciprocal Anti-Aβ1-42 Endpoint Total and Subclass Titers with varying Amounts of GM-CSF

Outbred Swiss-Webster mice were divided into groups of ten mice each. Each group received immunizations at week 0 and at week 3, with 30 μg of Aβ1-42 peptide each time. In the week 0 immunization, the first group received 50 μg of 529 SE; the second group received 50 μg of 529 SE plus 10 μg GM-CSF; the third group received 50 μg of 529 SE plus 5 μg GM-CSF; the fourth group received 50μg of 529 SE plus 2 μg GM-CSF; the fifth group received 50 μg of 529 SE plus 0.5 μg GM-CSF; the sixth group received 1% SE. In the week 3 immunization, the first through fifth groups received the same dose as the week 0 immunization, except that the 529 SE was reduced from 50 to 25 μg. The amount of SE received by the sixth group was increased from 1% in the week 0 immunization to 1.2% in the week 3 immunization. Mice were immunized subcutaneously in the rump with a total volume of 0.2 ml, divided equally into each of two sites at the base of the tail/rump.

Mice were bled at days 2, 20 and 35. Reciprocal endpoint anti-Aβ1-42 peptide IgG class and subclass titers were measured from individual serum (n=10) at week 5. The IgG endpoint results are given in Table 12. The IgG1 subclass results are given in Table 13. The IgG2a subclass results are given in Table 14.

TABLE 12
Anti-Aβ1-42 Week 5 IgG Endpoint Titers

| Adjuvants | Geometric Mean | Standard Error |
|---|---|---|
| 529 SE (50) | 6,119 | +/−3,103 |
| 529 SE (50) + GM- | 52,312 | +/−78,421 |
| CSF (10) | | |
| 529 SE (50) + GM- | 16,392 | +/−17,706 |
| CSF (5) | | |
| 529 SE (50) + GM- | 321,524 | +/−224,875 |
| CSF (2) | | |
| 529 SE (50) + GM- | 36,934 | +/−29,449 |
| CSF (0.5) | | |
| SE (1%) | 7,784 | +/−9,041 |

TABLE 13
Anti-Aβ1-42 Week 5 IgG1 Endpoint Titers

| Adjuvants | Geometric Mean | Standard Error |
|---|---|---|
| 529 SE (50) | 499 | +/−676 |
| 529 SE (50) + GM- | 1,424 | +/−2,468 |
| CSF (10) | | |
| 529 SE (50) + GM- | 3,407 | +/−5,653 |
| CSF (5) | | |
| 529 SE (50) + GM- | 18,328 | +/−8,067 |
| CSF (2) | | |
| 529 SE (50) + GM- | 3,526 | +/−17,606 |
| CSF (0.5) | | |
| SE (1%) | 2,556 | +/−5,615 |

TABLE 14
Anti-Aβ1-42 Week 5 IgG2a Endpoint Titers

| Adjuvants | Geometric Mean | Standard Error |
|---|---|---|
| 529 SE (50) | 1,386 | +/−5,173 |
| 529 SE (50) + GM- | 48,519 | +/−148,981 |
| CSF (10) | | |
| 529 SE (50) + GM- | 11,659 | +/−23,132 |
| CSF (5) | | |
| 529 SE (50) + GM- | 124,815 | +/−167,340 |
| CSF (2) | | |
| 529 SE (50) + GM- | 26,190 | +/−40,254 |
| CSF (0.5) | | |
| SE (1%) | 694 | +/−1,325 |

### Example 7

### Th-CTL and C4-V3 Peptide Immunization of Rhesus Monkeys

The following experiment was designed to directly compare a number of peptide and adjuvant combination formulations in a primate species (rhesus monkeys) in order to identify potential peptide/adjuvant combinations to move forward into human clinical trials. Specifically, the adjuvant formulation 529 SE with human GM-CSF was evaluated in comparison to incomplete Freund's adjuvant (IFA) in combination with (1) an SIV env-derived T-helper/SIV gag CTL peptide conjugate (ST1-p11C) having the following sequence:

| | |
|---|---|
| Arg Gln Ile Ile Asn Thr Trp His Lys Val Gly | (SEQ ID NO:3) |
| Lys Asn Val Tyr Leu Glu Gly Cys Thr Pro Tyr | |
| Asp Ile Asn Gln Met Leu; | |

or (2) an HIV-1 derived C4-V3 peptide conjugate (C4-V3_{89.6P}) having the following sequence:

| | |
|---|---|
| Lys Gln Ile Ile Asn Met Trp Gln Glu Val Gly | (SEQ ID NO:4) |
| Lys Ala Met Tyr Ala Thr Arg Pro Asn Asn Asn | |
| Thr Arg Glu Arg Leu Ser Ile Gly Pro Gly Arg | |
| Ala Phe Tyr Ala Arg Arg. | |

Study Design: A total of 8 animals was used for study, 4 Mamu-A*01+ and 4 Mamu-A*01− as described in Table 15.

TABLE 15
529 SE & GM-CSF vs. IFA

| | | | Immunogenic | |
|---|---|---|---|---|
| Group # | # Animals | Animal | composition | Adjuvant |
| 1 | 2 Mamu-A01+ | 95X009 | ST1-p11C | IFA |
| | | 93X021 | | |
| 2 | 2 Mamu-A01+ | 98N002 | ST1-p11C | 529 SE/GM-CSF |
| | | 98N008 | | |
| 3 | 2 Mamu-A01− | 98N007 | C4-V3_{89.6P} | IFA |
| | | 98N013 | | |
| 4 | 2 Mamu-A01− | 95X011 | C4-V3_{89.6P} | 529 SE/GM-CSF |
| | | 96X004 | | |

Group 1 animals received 0.5 ml of the Th-CTL peptide ST1-p11C (1.0 mg/ml) in a water in oil emulsion with 0.5 ml IFA in a total volume of 1.0 ml. The group 2 animals received 0.5 ml of ST1-p11C (1.0 mg/ml) combined with 250 μg of human GM-CSF, 50 μg of 529 SE with a final oil concentration of 1% in a total volume of 1.0 ml. Group 3 animals received 0.5 ml of the C4-V3_{89.6P} peptide (2.0 mg/ml) in a water in oil emulsion with 0.5 ml of IFA, final volume of 1.0 ml. Finally the group 4 animals received 0.5 ml C4-V3_{89.6P} peptide (2.0 mg/ml) combined with 250 μg of human GM-CSF, 50 μg of 529 SE with a final oil concentration of 1% in a total volume of 1.0 ml.

All animals were immunized by intramuscular injection on a schedule of 0, 4, and 8 weeks. Peripheral blood samples were drawn immediately before and 1 or 2 weeks after each immunization to monitor CTL induction by tetramer staining, p11C (Cys Thr Pro Tyr Asp Ile Asn Gln Met; SEQ ID NO:3, amino acids 19-27)—specific ELISPOT responses and bulk culture CTL responses (Groups 1 and 2) as well as for peptide specific antibody responses (Groups 1-4).

Safety and Tolerability

ST1-p11C+IFA: The ST1-p11C+IFA formulation which was administered by intramuscular injection at a single site three times in the group 1 animals was associated with significant injection site reactivity. One animal (93×021) developed a 1.5 cm sized abscess at the site of injection two weeks after the second immunization. The other animal (95×009) also developed a 2.0 cm sized abscess at the site of injection two weeks after the third immunization which broke through the skin and required dressing.

ST1-p11C+529 SE/GM-CSF: The ST1-p11C+529 SE/GM-CSF formulation which was administered by intramuscular injection at a single site three times in the group 2 animals was associated with minor adverse effects. Both of the group 2 animals vomited shortly after receiving the third immunization at week 8. No other adverse effects were noted.

C4-V3_{89.6P}+IFA: The C4-V3_{89.6P}+IFA formulation which was administered by intramuscular injection at a single site three times in the group 3 animals was associated with significant injection site reactivity. One animal (98n013) developed a 1.0 cm sized abscess at the site of injection one week after the second immunization. The other animal (98n007) also developed a 1.5 cm sized abscess at the site of injection one week after the second immunization. This animal's abscess required draining and dressing four weeks later.

C4-V3_{89.6P}+529 SE/GM-CSF: The C4-V3_{89.6P}+529 SE/GM-CSF formulation which was administered by intramuscular injection at a single site three times in the group 4 animals was associated with one minor adverse effect. One of the group 4 animals (95×011) vomited shortly after receiving the third immunization at week 8. No other adverse effects were noted.

In all of the group 1 and group 3 animals in which the animals received IFA as the adjuvant, significant injection site reactivities were seen. These results indicate that 0.5 mls of IFA is poorly tolerated when given by intramuscular injection at a single injection site. It is also worth noting that 3 of the 4 animals that received 529 SE/GM-CSF as the adjuvant at week 8 vomited shortly after being immunized. While the anesthetic used (ketamine) is known to be associated with vomiting, no other cases of animals vomiting were documented over the course of the study. At this time, insufficient evidence exists to attribute the animal's vomiting to any adverse effects associated with 529 SE/GM-CSF.

Results: Induction of Cellular Immune Responses (Groups 1 and 2)

Fresh Blood p11C-tetramer Staining: Prior to immunization, and one and two weeks post-immunization, freshly isolated peripheral blood from all the Mamu-A*01 positive animals (Groups 1 and 2) was screened for the presence of p11C-specific CD3⁺CD8⁺ T lymphocytes by soluble MHC Class I tetramer staining. As shown in Table 16, only one animal (93×021) which received the ST1-p11C peptide in combination with IFA, showed evidence of immunization-induced cellular immune responses in unstimulated peripheral blood.

TABLE 16
Percent p11C-tetramer staining and p11C-specific ELISPOT
responses in freshly isolated peripheral blood

| | | | | Week 5 | | Week 6 | |
|---|---|---|---|---|---|---|---|
| | | Week 0 | | 1 wk post 2^{nd} | | 2 wks post 2^{nd} | |
| | | Pre-immunization | | immunization | | immunization | |
| | | Fresh | ELISPOT | Fresh | ELISPOT | Fresh | ELISPOT |
| | | Bld | # SFC | Bld | # SFC | Bld | # SFC |
| Animal/ | | Tetramer | Per 10⁶ | Tetramer | Per 10⁶ | Tetramer | Per 10⁶ |
| (Group) | Formulation | staining^{a} | cells | staining | cells | staining | cells |
| 95x009(1) | ST1-p11C + IFA | 0.02 | 0.0 | 0.00 | 0.0 | 0.00 | 3.8 |
| 93x021(1) | ST1-p11C + IFA | 0.02 | Nd^{b} | 0.15 | 56.3 | 0.12 | 21.9 |
| 98n002(2) | ST1-p11C + 529SE/ | 0.00 | 0.6 | 0.05 | 0.0 | 0.01 | 6.3 |
| | GM-CSF | | | | | | |
| 98n008(2) | ST1-p11C + 529SE/ | 0.05 | 0.0 | 0.04 | 15.0 | 0.01 | 9.4 |
| | GM-CSF | | | | | | |
| | | Week 8 | | | | Week 10 | |
| | | 4 wks post 2^{nd} | | Week 91 wk post 3^{rd} | | 2 wks post 3^{rd} | |
| | | immunization | | immunization | | immunization | |
| | | Fresh | | | | Fresh | |
| Animal | Formulation | Bld | ELISPOT | Fresh Bld | ELISPOT | Bld | ELISPOT |
| 95x009(1) | ST1-p11C + IFA | 0.00 | Nd | 0.01 | 2.5 | 0.02 | 1.9 |
| 93x021(1) | ST1-p11C + IFA | 0.02 | Nd | 0.14 | Nd | 0.02 | Nd |
| 98n002(2) | ST1-p11C + 529SE/ | 0.06 | Nd | 0.00 | Nd | 0.00 | 3.8 |
| | GM-CSF | | | | | | |
| 98n008(2) | ST1-p11C + 529SE/ | 0.02 | Nd | 0.02 | Nd | 0.00 | 0.0 |
| | GM-CSF | | | | | | |

^{a}Reported as the percentage of fresh blood CD3⁺CD8⁺ lymphocytes staining positive with the p11C-tetramer;
ND, not done.
^{b}Nd = No data (in this and subsequent Tables).

P11C-specific ELISPOT responses: To further evaluate the induction of cellular immune responses in the Group 1 and 2 animals, freshly isolated peripheral blood lymphocytes were screened for the presence of p11C-specific CD3⁺CD8⁺ T lymphocytes by ELISPOT analysis. As shown in Table 16, only animal 93×021, which demonstrated detectable levels of p11C-specific CD8⁺ lymphocytes by fresh blood tetramer analysis, had detectable p11C-specific CD8⁺ T lymphocytes by ELISPOT analysis. In every case, a positive response by p11C-tetramer analysis was corroborated by a positive p11C-specific ELISPOT response.

p11C-specific cellular immune responses after in vitro peptide p11C stimulation: In an effort to increase the number of p11C-specific cells prior to analysis, freshly isolated peripheral blood lymphocytes were stimulated in vitro with peptide p11C and rhIL-2. After 14 days, the resulting effector cells were screened for p11C-tetramer binding as well as for functional p11C-specific lytic activity in a standard chromium release CTL assay. The results of the p11C-tetramer analysis and the functional CTL assays are shown in Table 17. Animal 93×021, which consistently showed p11C-specific immune responses in freshly isolated lymphocytes, demonstrated a very high level of tetramer binding and functional CTL activity one week after the second immunization. This indicated the induction of a very potent p11C-specific cellular immune response. In contrast to the results seen in the freshly isolated lymphocytes, one animal from Group 2 (98n008, ST1-p11C+529 SE/GM-CSF) began to show evidence of p11C-specific cellular immune responses two weeks after the second immunization. However, the p11C-specific immune response seen in the Group 2 animal was of a significantly lower magnitude than that seen in the responding animal from Group 1.

TABLE 17
Percent p11C-tetramer staining and functional p11C-
specific CTL responses after in vitro peptide p11C
stimulation

| | | | | Week 5 | | Week 6 | |
|---|---|---|---|---|---|---|---|
| | | Week 0 | | 1 wk post 2^{nd} | | 2 wks post 2^{nd} | |
| | | Pre-immunization | | immunization | | immunization | |
| Animal/ | | Tetramer | CTL | Tetramer | CTL | Tetramer | CTL |
| Group | Formulation | staining^{a} | 20:1 E:T^{b} | staining | 20:1 E:T | staining | 20:1 E:T |
| 95x009(1) | ST1-p11C + IFA | 0.03 | 0.6 | 0.23 | Nd | 0.27 | 0.0 |
| 93x021(1) | ST1-p11C + IFA | 0.03 | 0.0 | 34.31 | 66.3 | 15.15 | 4.69 |
| 98n002(2) | ST1-p11C + 529SE/ | 0.21 | 0.0 | Nd | Nd | 0.73 | Nd |
| | GM-CSF | | | | | | |
| 98n008(2) | ST1-p11C + 529SE/ | 0.16 | 0.0 | Nd | Nd | 5.84 | Nd |
| | GM-CSF | | | | | | |
| | | Week 8 | | Week 9 | | Week 10 | |
| | | 4 wks post 2^{nd} | | 1 wk post 3^{rd} | | 2 wks post 3^{rd} | |
| | | immunization | | immunization | | immunization | |
| | | | CTL | | | | CTL |
| Animal/ | | Tetramer | CTL | Tetramer | 20:1 | Tetramer | 20:1 |
| Group | Formulation | Staining | 20:1 E:T | Staining | E:T | Staining | E:T |
| 95x009(1) | ST1-p11C + IFA | Nd | Nd | 0.76 | 3.0 | 0.72 | 0.0 |
| 93x021(1) | ST1-p11C + IFA | Nd | Nd | 4.90 | 7.3 | Nd | Nd |
| 98n002(2) | ST1-p11C + 529SE/ | Nd | Nd | 0.07 | 5.7 | 0.39 | 0.0 |
| | GM-CSF | | | | | | |
| 99n008(2) | ST1-p11C + 529SE/ | Nd | Nd | 2.24 | 11.4 | 5.00 | 0.0 |
| | GM-CSF | | | | | | |

^{a}Reported as the percentage of CD3⁺CD8⁺ cultured cells staining positive with the p11c-tetramer.
^{b}Reported as the percent p11c-specific lysis (minus background) at an effector to target ratio (E:T) of 20:1.

Intracellular Cytokine Analysis: To further characterize the functional and phenotypic properties of the immunogen-induced peptide p11C-specific lymphocytes, the intracellular expression was monitored of the Th1 type cytokines INF-γ, TNFα, IL-2 and the Th2 type cytokine IL-4. Intracellular cytokine expression was monitored in peripheral blood lymphocytes after an initial in vitro stimulation in the presence of 10 μM peptide p11C and rhIL-2. The cultures were then maintained for 14 days with 40 U/ml IL-2. After two weeks, the cultured cells were stimulated with either media alone, or with 10 μM peptide p11C+anti-human CD28 and anti-human CD49d for one hour. After one hour, the cells were treated with Brefeldin A for an additional five hours to allow for intracellular cytokines to concentrate in the endoplasmic reticulum. Intracellular cytokine expression was then quantitated by flow cytometry (Tables 18 & 19).

As shown in Table 18, two weeks after in vitro peptide p11C stimulation, CD3⁺ peripheral blood lymphocytes from the group 1 animal 93×021 (ST1-p11C+IFA), demonstrated a low level of Th1 type cytokine expression, with less than 1.5% of all cells actively secreting INF-γ, TNFα, or IL-2. Approximately 8% of all CD3⁺ lymphocytes after in vitro peptide p11C stimulation were actively secreting the Th2 type cytokine IL-4, and the IL-4 secreting cells were found to be limited to the CD3⁺CD4⁺ lymphocyte subset. After a brief re-exposure to the peptide p11C, the p11C-tetramer+and CD3⁺CD8⁺ lymphocyte subsets were actively secreting the Th1 type cytokines INF-γ and TNFα, but could not be induced to secrete significantly increased levels of IL-2. After peptide p11C re-exposure, the secretion of the Th2 type cytokine IL-4 was unaffected.

TABLE 18
Intracellular cytokine analysis, group 1 animal 93x021,
two weeks post second immunization

| Lymphocyte | | INF-γ | | | TNFα | |
|---|---|---|---|---|---|---|
| Subset | Media | p11C | ^{a}S.I. | Media | p11C | S.I. |
| ^{b}P11C- | 977 | 27,612 | 28.3 | 90 | 20,342 | 226.0 |
| tetramer⁺ | | | | | | |
| ^{b}Bulk CD3⁺ | 7,628 | 65,567 | 8.6 | 12,256 | 51,361 | 4.2 |
| ^{b}CD3⁺CD4⁺ | 2,488 | 5,545 | 2.2 | 6,252 | 2,827 | 0.4 |
| ^{b}CD3⁺CD8⁺ | 5,273 | 60,573 | 11.5 | 6,865 | 48,439 | 7.1 |
| | | IL-2 | | | IL-4 | |
| | Media | p11C | S.I. | Media | p11C | S.I. |
| ^{b}P11C- | 751 | 2,004 | 2.7 | Nd | Nd | Nd |
| tetramer⁺ | | | | | | |
| ^{b}Bulk CD3⁺ | 2,879 | 6,463 | 2.2 | 78,069 | 90,563 | 1.2 |
| ^{b}CD3⁺CD4⁺ | 1,377 | 1,683 | 1.2 | 71,275 | 81,437 | 1.1 |
| ^{b}CD3⁺CD8⁺ | 1,502 | 4,783 | 3.2 | 6,794 | 9,126 | 1.3 |

TABLE 19
Intracellular cytokine analysis, group 2 animal 98n008,
one week post third immunization

| Lymphocyte | | INF-γ | | | TNFα | |
|---|---|---|---|---|---|---|
| Subset | Media | p11C | ^{a}S.I. | Media | p11C | S.I. |
| ^{b}P11C- | 545 | 560 | 1.0 | 748 | 454 | 0.0 |
| tetramer⁺ | | | | | | |
| ^{b}Bulk CD3⁺ | 6,829 | 10,489 | 1.5 | 3,402 | 13,443 | 4.0 |
| ^{b}CD3⁺CD4⁺ | 3,310 | 3,789 | 1.1 | 1,428 | 2,567 | 1.8 |
| ^{b}CD3⁺CD8⁺ | 3,189 | 6,825 | 2.1 | 2,587 | 11,324 | 4.4 |
| | | IL-2 | | | IL-4 | |
| | Media | p11C | S.I. | Media | p11C | S.I. |
| ^{b}P11C- | 77 | 456 | 5.9 | nd | nd | nd |
| tetramer⁺ | | | | | | |
| ^{b}Bulk CD3⁺ | 385 | 2,868 | 7.4 | 219,789 | 202,122 | 0.9 |
| ^{b}CD3⁺CD4⁺ | 1,379 | 1,761 | 1.3 | 170,804 | 158,175 | 0.9 |
| ^{b}CD3⁺CD8⁺ | 36 | 2,095 | 58.2 | 49,053 | 44,083 | 0.9 |

^{a}S.I., Stimulation index.
^{b}reported as the number of indicated cells staining positive for the indicated cytokine per 10⁶ CD3⁺ cells, minus background (isotype control) staining.

As shown in Table 19, two weeks after in vitro peptide p11C stimulation, CD3⁺ peripheral blood lymphocytes from the group 2 animal 98n008 (ST1-p11C+529 SE/GM-CSF) demonstrated a low level of Th1 type cytokine expression, with less than 1.0% of all cells actively secreting INF-γ, TNFα, or IL-2. Interestingly, approximately 20% of all CD3⁺ lymphocytes were actively secreting the Th2 type cytokine IL-4, a 2.5 fold increase in the number of IL-4 producing cells as compared to the Group 1 animal. As was the case with the Group 1 animal, the IL-4 secreting cells were found to be limited to the CD3⁺CD4⁺ lymphocyte subset. After a brief re-exposure to the peptide p11C, the p11C-tetramer⁺ and CD3⁺CD8⁺ lymphocyte subsets from the group 2 animal could be stimulated to secrete TNFα, but not INF-γ. In contrast to the Group 1 animal, after peptide re-exposure, a significant increase in IL-2 expression by CD3⁺CD8⁺ cells could be demonstrated. As was the case with the Group 1 animal, following the re-exposure to the peptide p11C, the secretion of the Th2 type cytokine IL-4 was unchanged.

Results: Immunogen-Induced Humoral Immune Responses (Groups 1 and 2):

To evaluate the induction of immunogen-specific humoral antibody responses, the anti-ST1-p11C ELISA antibody titers were measured in the serum of the group 1 and 2 animals immediately prior to immunization and 1 and 2 weeks after the second and third iummnizations. The results are summarized in Table 20.

TABLE 20
ELISA end-point titers of serum from rhesus monkeys
immunized with ST1-p11C (groups 1 and 2)

| | | | | ST1-p11C |
|---|---|---|---|---|
| Animal | Group | Formulation | Week | Ab titer^{a} |
| 95x009 | 1 | ST1-p11C + IFA | 5 | 12,800 |
| | | | 6 | 12,800 |
| | | | 9 | 6,400 |
| | | | 10 | 12,800 |
| 93x021 | 1 | ST1-p11C + IFA | 5 | 51,200 |
| | | | 6 | 102,400 |
| | | | 9 | 51,200 |
| | | | 10 | 51,200 |
| 98x002 | 2 | ST1-p11C + 529SE/GM-CSF | 5 | <50 |
| | | | 6 | <50 |
| | | | 9 | 1,600 |
| | | | 10 | <50 |
| 98n008 | 2 | ST1-p11C + 529SE/GM-CSF | 5 | 200 |
| | | | 6 | 200 |
| | | | 9 | 12,800 |
| | | | 10 | 6,400 |

^{a}Antibody end-point binding titers were determined as the reciprocal of the highest dilution of the plasma giving an OD reading of experimental/control (E/C) of >3.0.

Immunogen Induced Humoral Immune Responses (Groups 3 and 4):

To evalute the induction of immunogen-specific and adjuvant-specific humoral antibody responses, the anti-C4-V3_{89.6P} and anti-GM-CSF ELISA antibody titers were measured in the plasma of the group 3 and 4 animals immediately prior to immunization and one and two weeks after the second and third immunizations. The results are summarized in Table 21. The results indicate that peak plasma C4-V3_{89.6P} antibody titers were generated at one week after the second immunization in all the animals tested. Peak plasma antibody titers in the group 3 animals (C4-V3_{89.6P}+IFA) were several orders of magnitude higher than the peak plasma antibody titers seen in the group 4 (C4-V3_{89.6P}+529 SE/GM-CSF). The group 4 animals demonstrated low but detectable levels of anti-GM-CSF antibody titer that peaked one week after the immunization.

TABLE 21
ELISA end-point titers of plasma from rhesus monkeys
immunized with C4-V3_{89.6P} (groups 3 and 4)

| Animal/ | | | C4-V3 Ab | Anti-human |
|---|---|---|---|---|
| Group | Formulation | Week | titer^{a} | GM-CSF Ab titer |
| 98n007(3) | C4-V3_{89.6P} + IFA | 5 | 102,400 | <10 |
| | | 6 | 25,600 | <10 |
| | | 9 | 25,600 | <10 |
| | | 10 | 25,600 | <10 |
| 98n013(3) | C4-V3_{89.6P} + IFA | 5 | 12,800 | <10 |
| | | 6 | 6,400 | <10 |
| | | 9 | 12,800 | 10 |
| | | 10 | 12,800 | <10 |
| 96x004(4) | C4-V3_{89.6P} + | 5 | 6,400 | 320 |
| | 529SE/GM-CSF | 6 | 1,600 | 160 |
| | | 9 | 3,200 | 2,560 |
| | | 10 | 1,600 | 1,280 |
| 95x011(4) | C4-V3_{89.6P} + | 5 | 1,600 | 160 |
| | 529SE/GM-CSF | 6 | 800 | 80 |
| | | 9 | 1,600 | 1,280 |
| | | 10 | 1,600 | 1,280 |

^{a}Antibody end-point binding titers were determined as the reciprocal of the highest dilution of the plasma giving an OD reading of experimental/control (E/C) of >3.0.

The induction of neutralizing antibodies in the group 3 and 4 animals was also monitored; the results are summarized in Table 22. The results indicate that both group 3 animals and both group 4 animals had developed neutralizing antibodies that were capable of neutralizing the SHIV_{89.6} virus in vitro. The SHIV89.6 neutralizing antibody titers seen in the group 3 animals were generally higher than that seen in the group 4 animals. In addition, after the final immunization, the serum from the group three animals demonstrated a low level of neutralizing activity against the SHIV_{89.6P} strain of the virus, which is difficult to neutralize.

TABLE 22
Serum end-point neutralizing antibody titers from
rhesus monkeys immunized with C4-V3_{89.6P} (groups 3 and 4)

| | | | Neutralizing | |
|---|---|---|---|---|
| Animal/ | | | antibody to^{a} | |
| Group | Formulation | Week | SHIV_{89.6} | SHIV_{89.6P} |
| 98n007(3) | C4-V3_{89.6P} + IFA | 0 | <10 | <10 |
| | | 5 | 22 | <10 |
| | | 6 | 46 | <10 |
| | | 9 | 113 | 46 |
| | | 10 | 74 | 71 |
| 98n013(3) | C4-V3_{89.6P} + IFA | 0 | <10 | <10 |
| | | 5 | 12 | <10 |
| | | 6 | 19 | <10 |
| | | 9 | 36 | 18 |
| | | 10 | 22 | <10 |
| 96x004(4) | C4-V3_{89.6P + 529SE/GM-CSF} | 0 | <10 | <10 |
| | | 5 | <10 | <10 |
| | | 6 | <10 | <10 |
| | | 9 | 26 | <10 |
| | | 10 | <10 | <10 |
| 95x011(4) | C4-V3_{89.6P + 529SE/GM-CSF} | 0 | <10 | <10 |
| | | 5 | 11 | <10 |
| | | 6 | <10 | <10 |
| | | 9 | 19 | <10 |
| | | 10 | <10 | <10 |

^{a}Neutralizing antibody titers are the reciprocal serum dilution at which 50% of cells were protected from virus-induced killing as measured by neutral red uptake.

### Example 8

### Therapeutic Efficacy Study of PDAPP Transgenic Mice Treated with Aβ1-42 and Adjuvant(s)

The following experiment was designed to compare a number of adjuvant combination formulations in PDAPP transgenic mice to test the therapeutic efficacy of the Aβ1-42 peptide.

Study Design: Ten and a half to twelve and a half month old PDAPP transgenic mice (males and females) were divided into four groups of 40 mice, sorted such that each group was matched to each other as closely as possible for age, sex and transgenic parent. The groups were as described in Table 23:

TABLE 23
Transgenic Mice Treatment Groups

| | | | Aβ1- | N at | N at |
|---|---|---|---|---|---|
| Group | Adjuvant | Dose | 42 Dose | Start | End |
| 1 | MPL SE | 25 μg | 75/60 μg | 40 | 35 |
| 2 | MPL SE + GM-CSF | 25 μg/10 μg | 64/60 μg | 41 | 34 |
| 3 | 529 + GM-CSF | 25 μg/10 μg | 64/60 μg | 40 | 31 |
| 4 | PBS | na | na | 40 | 37 |

Aβ1-42 peptide was from Elan Pharmaceuticals, 529 SE and MPL™ SE were from Corixa, and murine GM-CSF was from Biosource. All mice received injections at weeks 0, 2, 4, 8, 12, 16, 20 and 24. Mice were bled 5-7 days post-injection, starting after the second injection. Groups 1, 2, and 3 were injected subcutaneously with a volume of 200 μl, while group 4 received 250 μl subcutaneous dosing. Animals were sacrificed at week 25 of the Study. Titers were obtained using a dilution which gives a value of 50% of the maximum optical density reading.

Results

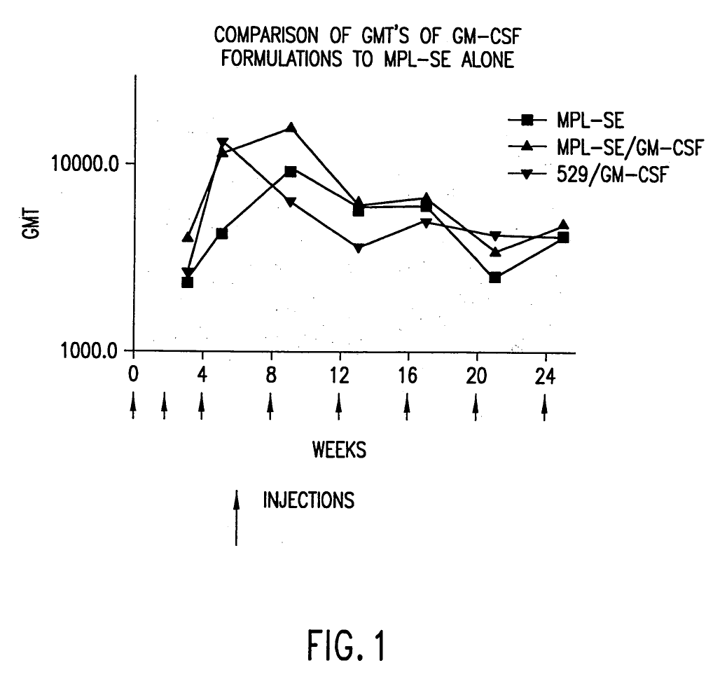

Immunogenicity and Antibody Response: All groups reached their peak geometric mean titer (GMT) of antibodies to the Aβ1-42 peptide at either the second (RC529+GM-CSF) or third bleed (MPL™ SE, MPL™SE+GM-CSF) (see FIG. 1). At peak GMT, MPL™ SE+GM-CSF was 16,400, while 529 SE+GM-CSF was 13,400 or approximately 1.5 times the MPL™ SE control of 9,700. However, the titers on the two GM-CSF-containing formulations (Groups 2 and 3) fell back to the approximate level of the MPL™ SE control, with final GMTs of MPL™ SE=4600, MPL™ SE+GM-CSF=5350, 529 SE+GM-CSF =4650.

To determine if the eventual decrease in titer of the two GM-CSF-containing formulations was due to an anti-GM-CSF antibody response, an ELISA was used to determine if anti-GM-CSF antibodies had formed over the course of the immunization. Sera from all animals receiving GM-CSF were titered against the murine GM-CSF used throughout this experiment. No evidence of anti-GM-CSF antibodies was found in any of the treated animals (data not shown).

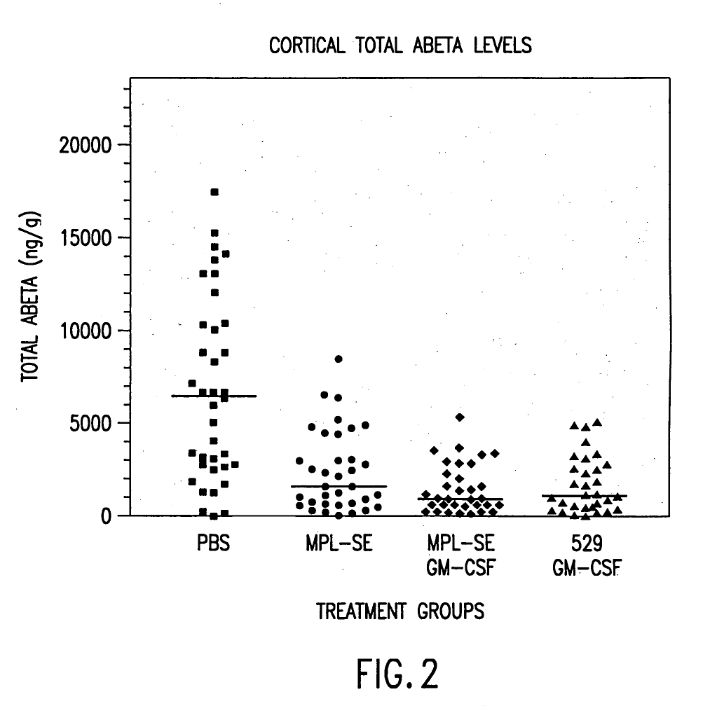

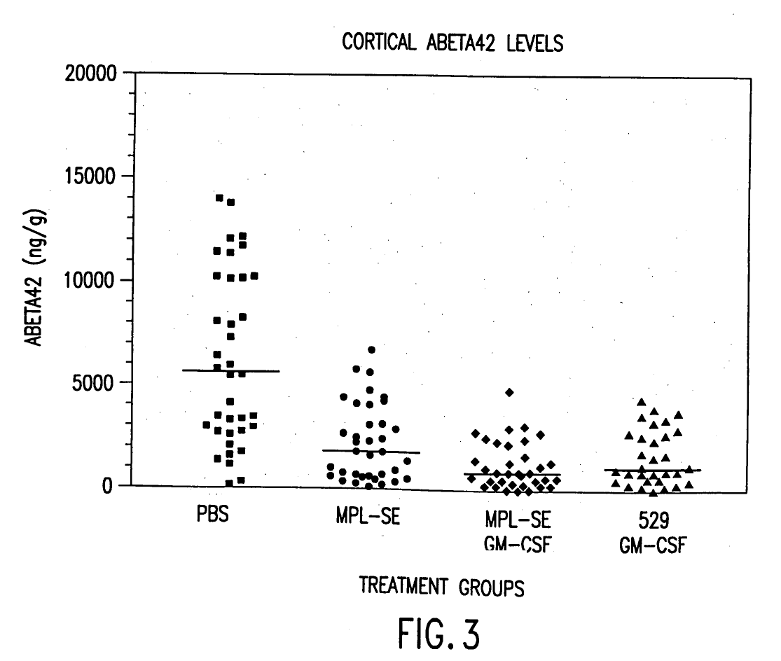

Brain Aβ Levels: All three treatment groups significantly reduced the accumulation of both total Aβ peptide (FIG. 2-individual results; Table 24-pooled results) and Aβ1-42 (FIG. 3-individual results; Table 25-pooled results) in the cortical region of the PDAPP mouse brain. Aβ ELISAs (total and 1-42 forms) were performed as previously described (54) using guanidine-solubilized brain homogenates. Statistical comparisons used the Mann-Whitney test of significance.

TABLE 24
Cortical Total Aβ Levels

| | | | MPL SE + | 529 SE + |
|---|---|---|---|---|
| | PBS | MPL SE | GM-CSF | GM-CSF |
| Median | 6,478 | 1,707 | 925 | 1,313 |
| (ng/g) | | | | |
| Range | 64-17,208 | 33-8,501 | 67-5,293 | 79-5,271 |
| % Reduction | — | 74 | 86 | 80 |
| P Value | — | <0.0001 | <0.0001 | <0.0001 |
| (M-W) | | | | |
| N | 37 | 35 | 34 | 31 |

TABLE 25
Cortical Aβ1-42 Levels

| | | | MPL SE + | 529 SE + |
|---|---|---|---|---|
| | PBS | MPL SE | GM-CSF | GM-CSF |
| Median | 5,609 | 1,799 | 779 | 1,127 |
| (ng/g) | | | | |
| Range | 284-14,004 | 10-6,715 | 43-4,824 | 29-4,442 |
| % Reduction | — | 68 | 86 | 80 |
| P Value | — | <0.0001 | <0.0001 | <0.0001 |
| (M-W) | | | | |
| N | 36 | 35 | 34 | 31 |

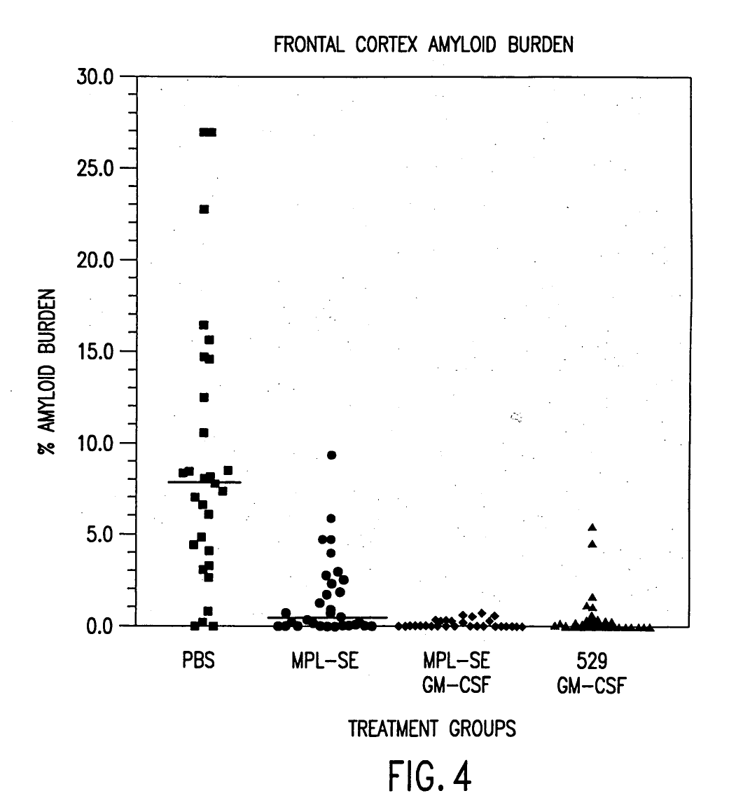

Amyloid Burden: The extent of amyloidosis was quantified in the frontal cortex using immunohistochemical methods as previously described (55). All three treatment groups showed a significant reduction in amyloid burden (FIG. 4-individual results; Table 26-pooled results).

TABLE 26
Frontal Cortex Amyloid Burden

| | | | MPL SE + | 529 SE + |
|---|---|---|---|---|
| | PBS | MPL SE | GM-CSF | GM-CSF |
| Median | 7.98 | 0.49 | 0.00 | 0.04 |
| (% AB) | | | | |
| Range | 0.00-27.37 | 0.00-9.63 | 0.00-.83 | 0.00-5.53 |
| % Reduction | — | 94 | 100 | 99.5 |
| P Value | — | <0.0001 | <0.0001 | <0.0001 |
| (M-W) | | | | |
| N | 29 | 33 | 29 | 30 |

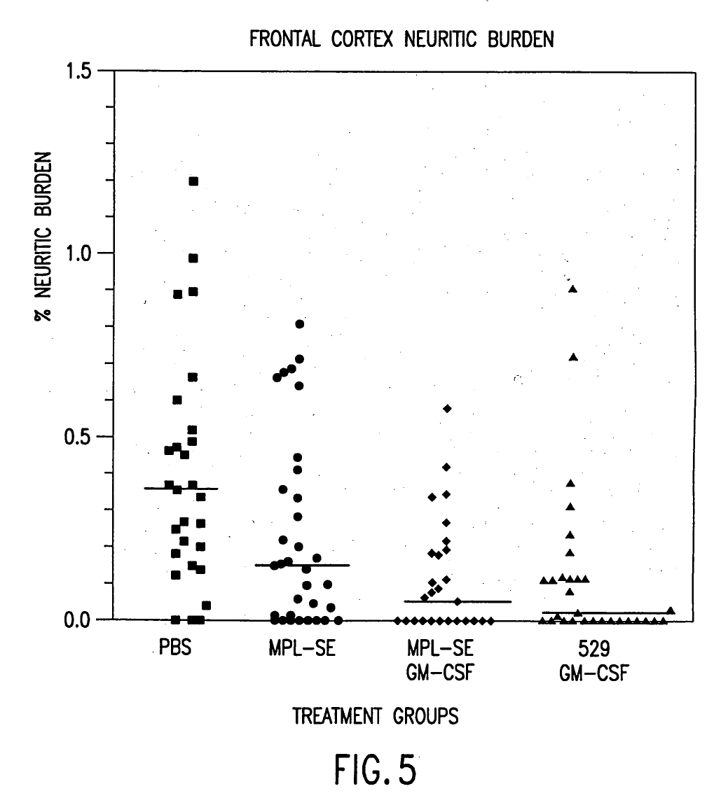

Neuritic Burden: The effect of treatment on the development of neuritic dystrophy in the frontal cortex was assessed immunohistochemical as previously described (55). All three treatment groups significantly reduced the extent of the neuritic burden relative to PBS control (FIG. 5-individual results; Table 27-pooled results).

TABLE 27
Frontal Cortex Neuritic Burden

| | | | MPL SE + | 529 SE + |
|---|---|---|---|---|
| | PBS | MPL SE | GM-CSF | GM-CSF |
| Median | 0.35 | 0.14 | 0.04 | 0.02 |
| (% NB) | | | | |
| Range | 0.00-1.21 | 0.00-0.82 | 0.00-0.60 | 0.00-0.92 |
| % Reduction | — | 60 | 88 | 95 |
| P Value | — | 0.0153 | <0.0001 | <0.0001 |
| (M-W) | | | | |
| N | 29 | 33 | 29 | 30 |

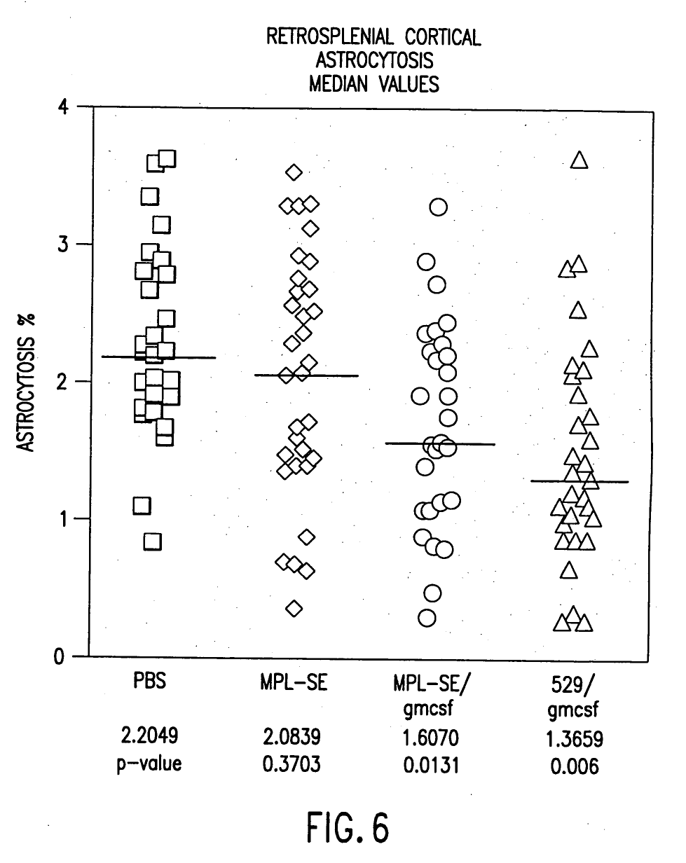

Astrocytosis: The extent of astrocytosis in the retrosplenial cortex was quantified as previously described (55). The treatment groups containing GM-CSF showed significantly reduced astrocytosis (FIG. 6).

### Example 9

### C4(E9V)-V3_{89.6P} Peptide Immunization of Cynomologous Macaques

The objective of this experiment was to evaluate the immunogenicity of an HIV-1 Env-derived peptide conjugate, C4(E9V)-V3_{89.6P}, administered with and without an adjuvant combination formulation of this invention in another primate species, cynomologous monkeys (Macaca fagscicularis). The C4-V3_{89.6P} peptide described in Example 7 was modified by changing the glutamic acid at amino acid residue 9 to valine. The resulting peptide conjugate, designated C4(E9V)-V3_{89.6P}, was used, and has the following sequence:

| | |
|---|---|
| Lys Gln Ile Ile Asn Met Trp Gln Val Val Gly | (SEQ |
| Lys Ala Met Tyr Ala Thr Arg Pro Asn Asn Asn | ID |
| Thr Arg Glu Arg Leu Ser Ile Gly Pro Gly Arg | NO: 5) |
| Ala Phe Tyr Ala Arg Arg | |

The glutamic acid to valine substitution (E9V) within the C4 region of the peptide increases the immunogenicity of the peptide above that of the unmodified sequence in the mouse model.

This HIV-1 Env-derived peptide is capable of eliciting humoral immune responses in mice. However, due to the difficulty of extrapolating mouse results to humans, it is necessary to test potential HIV-1 immunogenic compositions in non-human primates before proceeding into Phase I human clinical trials. In this experiment, both intramuscular (IM) and intranasal (IN) routes of administration were evaluated. Animals were immunized five times at weeks 0, 4, 8, 18 and 23. On a weekly basis through week 25, blood samples and cervicovaginal and mucosal washes were collected and analyzed for the presence of antibodies to the immunogenic composition.

Experimental Design: A total of eight cynomologous monkeys, four animals per group (Table 28), was used for the experiment. Group 1 received no adjuvant; Group 2 received the adjuvant formulation 529 SE plus GM-CSF. Animals are being housed and evaluated at an animal care facility.

TABLE 28
529 SE plus GM-CSF vs. no adjuvant

| Group # | Immunogen | Adjuvant | Route |
|---|---|---|---|
| 1 | C4(E9V)-V3_{89.6P} peptide | none | IN |
| 2 | C4(E9V)-V3_{89.6P} peptide | 529SE/GM-CSF | IM |

Immunizations: All intranasal immunizations were delivered with a 100 μl metered dose nasal spray device. All intramuscular injections were given in the quadriceps muscle with needle and syringe. All animals were immunized on a schedule of 0, 4, 8, 18 and 23 weeks.

Formulations:

Group 1: 1000 μg C4 (E9V)-V3_{89.6P} peptide in sterile normal saline, final volume 200 μl (100 μl each nostril).

Group 2: 1000 μg C4 (E9V)-V3_{89.6P} peptide, 50 μg 529 SE, and 250 μg human GM-CSF, final oil concentration 1%, final volume 1.0 ml (500 μl each quadriceps by IM injection).

Assays to monitor immunogen-induced immune responses:

Immediately before and after immunization, all animals were closely monitored for immunogen-induced humoral immune responses by the following assays:

(1) Serum anti-C4 (E9V)-V3_{89.6P} peptide IgG serum antibody titers by ELISA.

(2) Mucosal (cervicovaginal, nasal) anti-C4(E9V)-V3_{89.6P} peptide IgG antibody titers by ELISA.

Results:

Immunogen Safety and Tolerability:

The C4 (E9V)-V3_{89.6P} peptide when administered alone or in combination with the adjuvants 529 SE/GM-CSF was extremely well tolerated. For the animals immunized by the intramuscular route using needle and syringe, no adverse injection site reactivities were noted. All animals were closely monitored for changes in body temperature during the 24 hours immediately following each administration of the immunogen. At no time during the study, did any of the animals demonstrate abnormally elevated body temperature readings (data not shown).

Immunogen-specific Serum antibody responses:

Serum samples from all animals were obtained immediately prior to and one and two weeks after each immunization (through 25 weeks). Two weeks after the final immunization, all the serum samples were analyzed for the presence of anti-C4(E9V)-V3 peptide IgG antibodies. The intranasally immunized group 1 animals (C4(E9V)-V3_{89.6P} peptide alone) failed to demonstrate serum anti-C4(E9V)-V3_{89.6P} IgG antibody titers higher than pre-immune levels (FIG. 7). In contrast, the group 2 animals (IM administration of C4(E9V)-V3_{89.6P}+529 SE/GM-CSF) developed significant levels of serum C4(E9V)-V3-specific IgG antibodies (FIG. 7). The reported geometric mean endpoint titer is calculated using the lowest titer from each individual animal that is 3-fold over the reading for pooled naive serum at the same dilution.

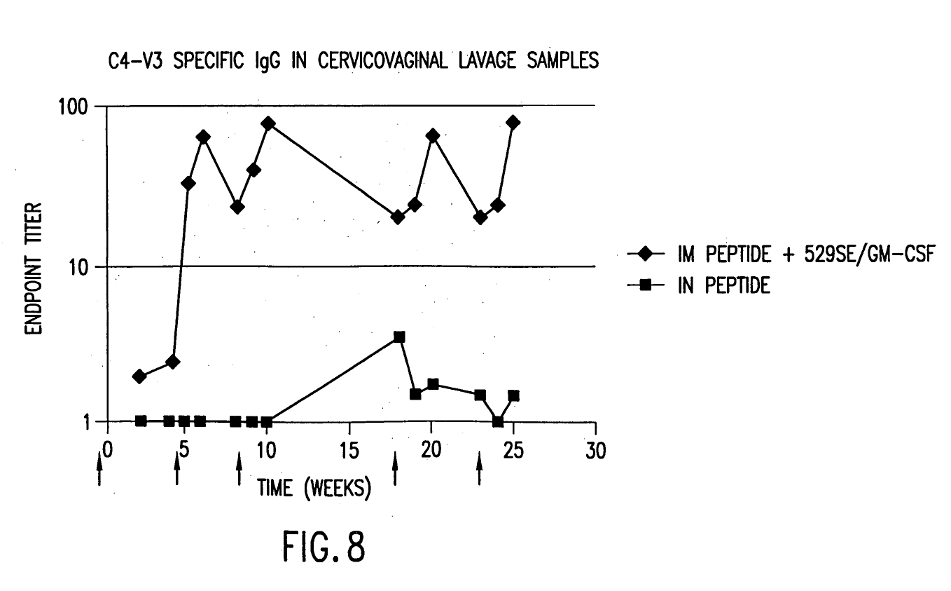

Group 1 animals (without adjuvant) had anti-C4(E9V)-V3_{89.6P} IgG antibody titers in cervicovaginal lavage samples that were higher than pre-immune levels only after the fourth immunization, but declined thereafter (FIG. 8). In contrast, group 2 animals (with adjuvant) had anti- C4(E9V)-V3_{89.6P} IgG antibody titers in cervicovaginal lavage samples that were higher than pre-immune levels after the first immunization and increased after each subsequent immunization (although there was some later drop-off in each case) (FIG. 8).

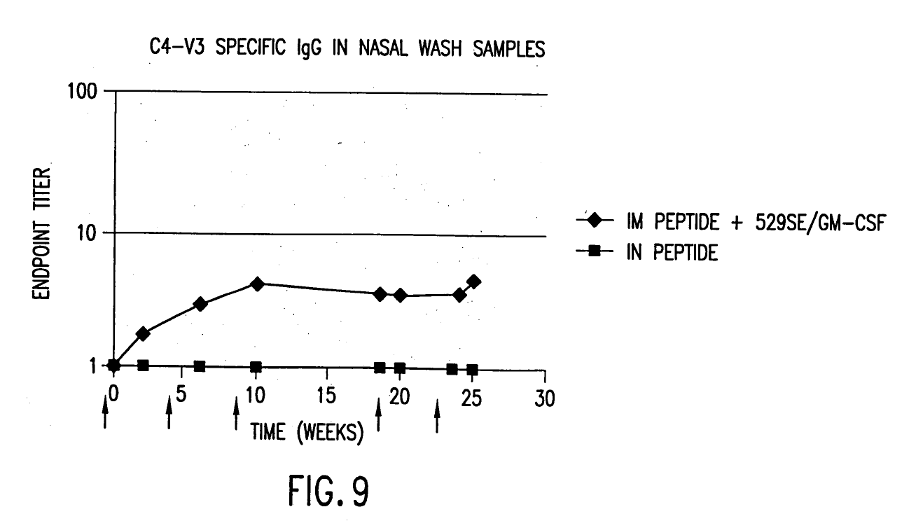

Group 1 animals (without adjuvant) failed to demonstrate anti-C4(E9V)-V3_{89.6P} IgG antibody titers in nasal washes higher than pre-immune levels (FIG. 9). In contrast, group 2 animals (with adjuvant) developed significant levels of anti-C4(E9V)-V3_{89.6P} IgG antibody titers in nasal wash samples (FIG. 9).

### Bibliography

1. Mosmann, T. R., et al., J. Immunol., 136, 2348-2357 (1986).

2. U.S. Pat. No. 5,057,540

3. U.S. Pat. No. 6,113,918.

4. Ahlers, J. D., et al., J. Immunol., 158, 3947-3958 (1997).

5. Scharton-Kersten, T., et al., J. Immunol. 154, 5320-5330 (1995).

6. Ghalib, H. W., et al., J. Immunol., 154, 4623-4629 (1995).

7. Murray, H. W., and Hariprashad, J.,. J. Exp. Med., 181, 387-391 (1995).

8. U.S. Pat. No. 5,571,515.

9. U.S. Pat. No. 5,723,127.

10. U.S. Pat. No. 5,976,539.

11. Finkelman, F. D., and Holmes, J., Ann. Rev. Immunol., 8, 303-333 (1990).

12. Snapper, C. M., et al., J. Exp. Med., 175, 1367-1371 (1992).

13. Kobayashi, M., et al., J. Exp. Med., 170, 827-845 (1989).

14. Published International Patent Application Number WO 90/05147.

15. U.S. Pat. No. 5,078,996.

16. U.S. Pat. No. 5,229,496.

17. U.S. Pat. No. 5,073,627.

18. Alderson, M. R., et al., J. Exp. Med., 178, 669-674 (1993).

19. Snapper, C. M., et al., J. Immunol., 154, 5842-5850 (1995).

20. U.S. Pat. No. 5,679,356.

21. Published International-Patent Application Number WO 00/69456.

22. U.S. Pat. No. 5,013,548.

23. U.S. Pat. No. 5,019,387.

24. Charbit, A., et al., Vaccine, 11, 1221-1228 (1993).

25. Natuk, R. J., et al., AIDS Res. Hum. Retroviruses, 9, 395-404 (1993).

26. Johnson, R. P., et al., J. Virol., 68, 3145-3153 (1994).

27. Fuller, D. H., et al., AIDS Res. Hum. Retroviruses, 10, 1433-1441 (1994).

28. Berzofsky, J. A., et al., J. Clin. Invest., 88, 876-884 (1991).

29. Palker, T. J., et al., J. Immunol., 142, 3612-3619 (1989).

30. Hart, M. K., et al., J. Immunol., 145, 2677-2685 (1990).

31. Haynes, B. F., et al., J. Immunol., 151, 1646-1653 (1993).

32. Hart, M. K., et al., Proc. Natl. Acad. Sci., USA, 88, 9448-9452 (1991).

33. Bartlett, J. A., et al., AIDS, 12, 1291-1300 (1998).

34. Haynes, B. F., et al., AIDS Res. Hum. Retroviruses, 11, 211-221 (1998).

35. U.S. Pat. No. 5,861,243.

36. U.S. Pat. No. 5,932,218.

37. U.S. Pat. No. 5,939,074.

38. U.S. Pat. No. 5,993,819.

39. U.S. Pat. No. 6,037,135.

40. Published European Patent Application Number 671,947.

41. U.S. Pat. No. 6,024,965.

42. Miller, M. D., et al., J. Immunol., 147, 320-329 (1991).

43. Kuroda, M. J., et al., J. Exp. Med., 187, 1373-1381 (1998).

44. Liao, H-X, et al., J. Virol., 74, 254-263 (2000).

45. Staats, H. F., et al., J. Immunol., 157, 462-472 (1996).

46. Porgador, A., et al., J. Immunol., 158, 834-841 (1997).

47. Published International Patent Application Number WO 99/27944.

48. U.S. Pat. No. 4,666,829.

49. Games, D., et al., Nature, 373, 523-527 (1995).

50. Published International Patent Application Number WO 98/20734.

51. U.S. Pat. No. 5,830,877.

52. Published International Patent Application Number WO 99/51259.

53. U.S. Pat. No. 5,593,972.

54. Johnson-Wood, D., et al., Proc. Natl. Acad. Sci., USA, 94, 1550-1555 (1997).

55. Schenk, D., et al., Nature, 400, 173-177 (1999).

What is claimed is:

1. An antigenic composition comprising a selected antigen from a pathogenic virus, bacterium, fungus or parasite, or from a cancer cell or tumor cell, or from an allergen, or from a self molecule, and an effective adjuvanting amount of the combination of: (1) an aminoalkyl glucosamine phosphate compound (AGP), or a derivative or analog thereof, and (2) a cytokine or lymphokine, or an agonist to said cytokine or lymphokine, wherein the combination of adjuvants enhances the immune response in a vertebrate host to said antigen.

2. The antigenic composition of claim 1 where the selected antigen is a polypeptide, peptide or fragment derived from a protein.

3. The antigenic composition of claim 1 where the AGP is used in the form of a stable oil-in-water emulsion.

4. The antigenic composition of claim 1 where the cytokine or lymphokine is selected from the group consisting of granulocyte macrophage colony stimulating factor and interleukin-12.

5. The antigenic composition of claim 4 where the cytokine or lymphokine is granulocyte macrophage colony stimulating factor.

6. The antigenic composition of claim 5 where the AGP is used in the form of a stable oil-in-water emulsion.

7. The antigenic composition of claim 4 where the cytokine or lymphokine is interleukin-12.

8. The antigenic composition of claim 7 where the AGP is used in the form of a stable oil-in-water emulsion.

9. The antigenic composition of claim 1 which further comprises a diluent or carrier.

10. The antigenic composition of claim 9 where the AGP is used in the form of a stable oil-in-water emulsion.

11. The antigenic composition of claim 1 where the AGP is:
2-[(R)-3-Tetradecanoyloxytetradecanoylamino]ethyl 2-Deoxy-4-O-phosphono-3-O-[(R)-3-tetradecanoyoxytetradecanoyl]-2-[(R)-3-tetradecanoyoxytetradecanoylamino]-β-D-glucopyranoside (529).

12. The antigenic composition of claim 1 where the selected antigen is from human immunodeficiency virus (HIV).

13. The antigenic composition of claim 12 where the selected HIV antigen is an HIV protein, polypeptide, peptide or fragment derived from said protein.

14. The antigenic composition of claim 13 where the selected antigens are HIV peptides selected from the group consisting of peptides having the amino acid sequence:

| | |
|---|---|
| Lys Gln Ile Ile Asn Met Trp Gln Glu Val Gly Lys Ala Met | (SEQ ID NO: 1) |
| Tyr Ala Cys Thr Arg Pro Asn Tyr Asn Lys Arg Lys Arg Ile | |
| His Ile Gly Pro Gly Arg Ala Phe Tyr Thr Thr Lys; | |
| Lys Gln Ile Ile Asn Met Trp Gln Glu Val Gly Lys Ala Met | (SEQ ID NO: 2) |
| Tyr Ala Thr Arg Pro Asn Tyr Asn Lys Arg Lys Arg Ile His | |
| Ile Gly Pro Gly Arg Ala Phe Tyr Thr Thr Lys; | |
| Arg Gln Ile Ile Asn Thr Trp His Lys Val Gly Lys Asn Val | (SEQ ID NO: 3) |
| Tyr Leu Glu Gly Cys Thr Pro Tyr Asp Ile Asn Gln Met Leu; | |
| Lys Gln Ile Ile Asn Met Trp Gln Glu Val Gly Lys Ala Met | (SEQ ID NO: 4) |
| Tyr Ala Thr Arg Pro Asn Asn Asn Thr Arg Glu Arg Leu Ser | |
| Ile Gly Pro Gly Arg Ala Phe Tyr Ala Arg Arg; | |
| and | |
| Lys Gln Ile Ile Asn Met Trp Gln Val Val Gly Lys Ala Met | (SEQ ID NO: 5) |
| Tyr Ala Thr Arg Pro Asn Asn Asn Thr Arg Glu Arg Leu Ser | |
| Ile Gly Pro Gly Arg Ala Phe Tyr Ala Arg Arg. | |

15. The antigenic composition of claim 14 wherein the HIV peptide is the peptide having the amino acid sequence:

| | |
|---|---|
| Lys Gln Ile Ile Asn Met Trp Gln Glu Val Gly Lys Ala Met | (SEQ ID NO: 1) |
| Tyr Ala Cys Thr Arg Pro Asn Tyr Asn Lys Arg Lys Arg Ile | |
| His Ile Gly Pro Gly Arg Ala Phe Tyr Thr Thr Lys. | |

16. The antigenic composition of claim 14 wherein the HIV peptide is the peptide having the amino acid sequence:

| | |
|---|---|
| Lys Gln Ile Ile Asn Met Trp Gln Glu Val Gly Lys Ala Met | (SEQ ID NO: 2) |
| Tyr Ala Thr Arg Pro Asn Tyr Asn Lys Arg Lys Arg Ile His | |
| Ile Gly Pro Gly Arg Ala Phe Tyr Thr Thr Lys. | |

17. The antigenic composition of claim 14 wherein the HIV peptide is the peptide having the amino acid sequence:

| | |
|---|---|
| Arg Gln Ile Ile Asn Thr Trp His Lys Val Gly Lys Asn Val | (SEQ ID NO: 3) |
| Tyr Leu Glu Gly Cys Thr Pro Tyr Asp Ile Asn Gln Met Leu. | |

18. The antigenic composition of claim 14 wherein the HIV peptide is-the peptide having the amino acid sequence:

| | |
|---|---|
| Lys Gln Ile Ile Asn Met Trp Gln Glu Val Gly Lys Ala Met | (SEQ ID NO: 4) |
| Tyr Ala Thr Arg Pro Asn Asn Asn Thr Arg Glu Arg Leu Ser | |
| Ile Gly Pro Gly Arg Ala Phe Tyr Ala Arg Arg. | |

19. The antigenic composition of claim 14 wherein the HIV peptide is the peptide having the amino acid sequence:

| | |
|---|---|
| Lys Gln Ile Ile Asn Met Trp Gln Val Val Gly Lys Ala Met. | (SEQ ID NO: 5) |
| Tyr Ala Thr Arg Pro Asn Asn Asn Thr Arg Glu Arg Leu Ser | |
| Ile Gly Pro Gly Arg Ala Phe Tyr Ala Arg Arg | |

20. The antigenic composition of claim 12 where the AGP is used in the form of a stable oil-in-water emulsion.

21. The antigenic composition of claim 12 where the cytokine or lymphokine is selected from the group consisting of granulocyte macrophage colony stimulating factor and interleukin-12.

22. The antigenic composition of claim 21 where the cytokine or lymphokine is granulocyte macrophage colony stimulating factor.

23. The antigenic composition of claim 22 where the AGP is used in the form of a stable oil-in-water emulsion.

24. The antigenic composition of claim 21 where the cytokine or lymphokine is interleukin-12.

25. The antigenic composition of claim 24 where the AGP is used in the form of a stable oil-in-water emulsion.

26. The antigenic composition of claim 12 which further comprises a diluent or carrier.

27. The antigenic composition of claim 26 where the AGP is used in the form of a stable oil-in-water emulsion.

28. The antigenic composition of claim 12 where the AGP is 529.

29. A method for increasing the ability of an antigenic composition containing a selected antigen from a pathogenic virus, bacterium, fungus or parasite to elicit the immune response of a vertebrate host, which comprises administering to said host an antigenic composition of claim 1.

30. A method for increasing the ability of an antigenic composition containing a selected antigen from a pathogenic virus, bacterium, fungus or parasite to elicit the immune response of a vertebrate host, which comprises administering to said host an antigenic composition of claim 9.

31. A method for increasing the ability of an antigenic composition containing an HIV antigen to elicit the immune response of a vertebrate host, which comprises administering to said host an antigenic composition of claim 12.

32. A method for increasing the ability of an antigenic composition containing an HIV antigen to elicit the immune response of a vertebrate host, which comprises administering to said host an antigenic composition of claim 26.

33. The method of claim 32 where the selected antigens are HIV peptides selected from the group consisting of peptides having the amino acid sequence:

| | |
|---|---|
| Lys Gln Ile Ile Asn Met Trp Gln Glu Val Gly Lys Ala Met | (SEQ ID NO: 1) |
| Tyr Ala Cys Thr Arg Pro Asn Tyr Asn Lys Arg Lys Arg Ile | |
| His Ile Gly Pro Gly Arg Ala Phe Tyr Thr Thr Lys; | |
| Lys Gln Ile Ile Asn Met Trp Gln Glu Val Gly Lys Ala Met | (SEQ ID NO: 2) |
| Tyr Ala Thr Arg Pro Asn Tyr Asn Lys Arg Lys Arg Ile His | |
| Ile Gly Pro Gly Arg Ala Phe Tyr Thr Thr Lys; | |
| Arg Gln Ile Ile Asn Thr Trp His Lys Val Gly Lys Asn Val | (SEQ ID NO: 3) |
| Tyr Leu Glu Gly Cys Thr Pro Tyr Asp Ile Asn Gln Met Leu; | |
| Lys Gln Ile Ile Asn Met Trp Gln Glu Val Gly Lys Ala Met | (SEQ ID NO: 4) |
| Tyr Ala Thr Arg Pro Asn Asn Asn Thr Arg Glu Arg Leu Ser | |
| Ile Gly Pro Gly Arg Ala Phe Tyr Ala Arg Arg; | |
| and | |
| Lys Gln Ile Ile Asn Met Trp Gln Val Val Gly Lys Ala Met | (SEQ ID NO: 5) |
| Tyr Ala Thr Arg Pro Asn Asn Asn Thr Arg Glu Arg Leu Ser | |
| Ile Gly Pro Gly Arg Ala Phe Tyr Ala Arg Arg. | |

34. The method of claim 33 wherein the HIV peptide is the peptide having the amino acid sequence:

| | |
|---|---|
| Lys Gln Ile Ile Asn Met Trp Gln Glu Val Gly Lys Ala Met | (SEQ ID NO: 1) |
| Tyr Ala Cys Thr Arg Pro Asn Tyr Asn Lys Arg Lys Arg Ile | |
| His Ile Gly Pro Gly Arg Ala Phe Tyr Thr Thr Lys. | |

35. The method of claim 33 wherein the HIV peptide is the peptide having the amino acid sequence:

| | |
|---|---|
| Lys Gln Ile Ile Asn Met Trp Gln Glu Val Gly Lys Ala Met | (SEQ ID NO: 2) |
| Tyr Ala Thr Arg Pro Asn Tyr Asn Lys Arg Lys Arg Ile His | |
| Ile Gly Pro Gly Arg Ala Phe Tyr Thr Thr Lys. | |

36. The method of claim 33 wherein the HIV peptide is the peptide having the amino acid sequence:

| | |
|---|---|
| Arg Gln Ile Ile Asn Thr Trp His Lys | (SEQ ID NO:3) |
| Val Gly Lys Asn Val Tyr Leu Glu Gly | |
| Cys Thr Pro Tyr Asp Ile Asn Gln Met | |
| Leu. | |

37. The method of claim 33 wherein the HIV peptide is the peptide having the amino acid sequence:

| | |
|---|---|
| Lys Gln Ile Ile Asn Met Trp Gln Glu | (SEQ ID NO:4) |
| Val Gly Lys Ala Met Tyr Ala Thr Arg | |
| Pro Asn Asn Asn Thr Arg Glu Arg Leu | |
| Ser Ile Gly Pro Gly Arg Ala Phe Tyr | |
| Ala Arg Arg. | |

38. The method of claim 33 wherein the HIV peptide is the peptide having the amino acid sequence:

| | |
|---|---|
| Lys Gln Ile Ile Asn Met Trp Gln Val | (SEQ ID NO:5) |
| Val Gly Lys Ala Met Tyr Ala Thr Arg | |
| Pro Asn Asn Asn Thr Arg Glu Arg Leu | |
| Ser Ile Gly Pro Gly Arg Ala Phe Tyr | |
| Ala Arg Arg. | |

39. A method for increasing the ability of an antigenic composition containing a selected antigen from a pathogenic virus, bacterium, fungus or parasite to elicit cytotoxic T lymphocytes in a vertebrate host, which comprises administering to said host an antigenic composition of claim 1.

40. A method for increasing the ability of an antigenic composition containing a selected antigen from a pathogenic virus, bacterium, fungus or parasite to elicit cytotoxic T lymphocytes in a vertebrate host, which comprises administering to said host an antigenic composition of claim 9.

41. A method for increasing the ability of an antigenic composition containing an HIV antigen to elicit cytotoxic T lymphocytes in a vertebrate host, which comprises administering to said host an antigenic composition of claim 12.

42. A method for increasing the ability of an antigenic composition containing an HIV antigen to elicit cytotoxic T lymphocytes in a vertebrate host, which comprises administering to said host an antigenic composition of claim 26.

43. The method of claim 42 where the selected antigens are HIV peptides selected from the group consisting of peptides having the amino acid sequence:

| | |
|---|---|
| Lys Gln Ile Ile Asn Met Trp Gln Glu | (SEQ ID NO:1) |
| Val Gly Lys Ala Met Tyr Ala Cys Thr | |
| Arg Pro Asn Tyr Asn Lys Arg Lys Arg | |
| Ile His Ile Gly Pro Gly Arg Ala Phe | |
| Tyr Thr Thr Lys; | |
| Lys Gln Ile Ile Asn Met Trp Gln Glu | (SEQ ID NO:2) |
| Val Gly Lys Ala Met Tyr Ala Thr Arg | |
| Pro Asn Tyr Asn Lys Arg Lys Arg Ile | |
| His Ile Gly Pro Gly Arg Ala Phe Tyr | |
| Thr Thr Lys; | |
| Arg Gln Ile Ile Asn Thr Trp His Lys | (SEQ ID NO:3) |
| Val Gly Lys Asn Val Tyr Leu Glu Gly | |
| Cys Thr Pro Tyr Asp Ile Asn Gln Met | |
| Leu; | |
| Lys Gln Ile Ile Asn Met Trp Gln Glu | (SEQ ID NO:4) |
| Val Gly Lys Ala Met Tyr Ala Thr Arg | |
| Pro Asn Asn Asn Thr Arg Glu Arg Leu | |
| Ser Ile Gly Pro Gly Arg Ala Phe Tyr | |
| Ala Arg Arg; | |
| and | |
| Lys Gln Ile Ile Asn Met Trp Gln Val | (SEQ ID NO:5) |
| Val Gly Lys Ala Met Tyr Ala Thr Arg | |
| Pro Asn Asn Asn Thr Arg Glu Arg Leu | |
| Ser Ile Gly Pro Gly Arg Ala Phe Tyr | |
| Ala Arg Arg. | |

44. The method of claim 43 wherein the HIV peptide is the peptide having the amino acid sequence:

| | |
|---|---|
| Lys Gln Ile Ile Asn Met Trp Gln Glu | (SEQ ID NO:1) |
| Val Gly Lys Ala Met Tyr Ala Cys Thr | |
| Arg Pro Asn Tyr Asn Lys Arg Lys Arg | |
| Ile His Ile Gly Pro Gly Arg Ala Phe | |
| Tyr Thr Thr Lys. | |

45. The method of claim 43 wherein the HIV peptide is the peptide having the amino acid sequence:

| | |
|---|---|
| Lys Gln Ile Ile Asn Met Trp Gln Glu | (SEQ ID NO:2) |
| Val Gly Lys Ala Met Tyr Ala Thr Arg | |
| Pro Asn Tyr Asn Lys Arg Lys Arg Ile | |
| His Ile Gly Pro Gly Arg Ala Phe Tyr | |
| Thr Thr Lys. | |

46. The method of claim 43 wherein the HIV peptide is the peptide having the amino acid sequence:

| | |
|---|---|
| Arg Gln Ile Ile Asn Thr Trp His Lys | (SEQ ID NO:3) |
| Val Gly Lys Asn Val Tyr Leu Glu Gly | |
| Cys Thr Pro Tyr Asp Ile Asn Gln Met | |
| Leu. | |

47. The method of claim 43 wherein the HIV peptide is the peptide having the amino acid sequence:

| | |
|---|---|
| Lys Gln Ile Ile Asn Met Trp Gln Glu | (SEQ ID NO:4) |
| Val Gly Lys Ala Met Tyr Ala Thr Arg | |
| Pro Asn Asn Asn Thr Arg Glu Arg Leu | |
| Ser Ile Gly Pro Gly Arg Ala Phe Tyr | |
| Ala Arg Arg. | |

48. The method of claim 43 wherein the HIV peptide is the peptide having the amino acid sequence:

| | |
|---|---|
| Lys Gln Ile Ile Asn Met Trp Gln Val | (SEQ ID NO:5) |
| Val Gly Lys Ala Met Tyr Ala Thr Arg | |
| Pro Asn Asn Asn Thr Arg Glu Arg Leu | |
| Ser Ile Gly Pro Gly Arg Ala Phe Tyr | |
| Ala Arg Arg. | |

49. A method for increasing the ability of an antigenic composition containing a selected cancer antigen or tumor-associated antigen from a cancer cell or tumor cell to elicit a therapeutic or prophylactic anti-cancer effect in a vertebrate host, which comprises administering to said host an antigenic composition comprising said selected cancer antigen or tumor-associated antigen from a cancer cell or tumor cell, and an effective adjuvanting amount of the combination of: (1) an AGP, or a derivative or analog thereof, and (2) a cytokine or lymphokine, or an agonist to said cytokine or lymphokine.

50. A method for increasing the ability of an antigenic composition containing a selected allergen to moderate an allergic response in a vertebrate host, which comprises administering to said host an antigenic composition comprising said allergen, and an effective adjuvanting amount of the combination of: (1) an AGP, or a derivative or analog thereof, and (2) a cytokine or lymphokine, or an agonist to said cytokine or lymphokine.

51. An antigenic composition containing a selected antigen from a molecule or portion thereof which represents those produced by a host in an undesired manner, amount or location so as to reduce such an undesired effect, by including an effective adjuvanting amount of the combination of: (1) an AGP, or a derivative or analog thereof, and (2) a cytokine or lymphokine, or an agonist to said cytokine or lymphokine.

52. The antigenic composition of claim 51, where the selected antigen is a polypeptide, peptide or fragment derived from amyloid precursor protein, or an antibody thereto.

53. The antigenic composition of claim 52, where the selected antigen is the Aβ peptide, which is an internal, 39-43 amino acid fragment of amyloid precursor protein, or a fragment of the Aβ peptide.

54. The antigenic composition of claim 53, where the selected antigen is the Aβ peptide having the amino acid sequence:

| | |
|---|---|
| Asp Ala Glu Phe Arg His Asp Ser Gly | (SEQ ID NO:6) |
| Tyr Glu Val His His Gln Lys Leu Val | |
| Phe Phe Ala Glu Asp Val Gly Ser Asn | |
| Lys Gly Ala Ile Ile Gly Leu Met Val | |
| Gly Gly Val Val Ile Ala. | |

55. The antigenic composition of claim 54, where the AGP is 529.

56. An method for increasing the ability of an antigenic composition containing a selected antigen from a molecule or portion thereof which represents those produced by a host in an undesired manner, amount or location so as to reduce such an undesired effect, by including an effective adjuvanting amount of the combination of: (1) an AGP, or a derivative or analog thereof, and (2) a cytokine or lymphokine, or an agonist to said cytokine or lymphokine.

57. A method for increasing the ability of an antigenic composition to prevent or treat disease characterized by amyloid deposition in a vertebrate host, which comprises administering to said host a polypeptide, peptide or fragment derived from amyloid precursor protein, or an antibody thereto, and an effective adjuvanting amount of the combination of: (1) an AGP, or a derivative or analog thereof, and (2) a cytokine or lymphokine, or an agonist to said cytokine or lymphokine.

58. The method of claim 57 where the selected antigen is the Aβ peptide, which is an internal, 39-43 amino acid fragment of amyloid precursor protein, or a fragment of the Aβ peptide.

59. The method of claim 58 where the selected antigen is the Aβ peptide having the amino acid sequence:

| | |
|---|---|
| Asp Ala Glu Phe Arg His Asp Ser Gly Tyr Glu Val His His. | (SEQ ID NO: 6) |
| Gln Lys Leu Val Phe Phe Ala Glu Asp Val Gly Ser Asn Lys | |
| Gly Ala Ile Ile Gly Leu Met Val Gly Gly Val Val Ile Ala | |

60. The method of claim 59 where the AGP is 529.

61. An adjuvant formulation comprising the combination of: (1) an aminoalkyl glucosamine phosphate compound (AGP), or a derivative or analog thereof, and (2) a cytokine or lymphokine, or an agonist to said cytokine or lymphokine.

62. The adjuvant formulation of claim 61 where the AGP is used in the form of a stable oil-in-water emulsion.

63. The adjuvant formulation of claim 61 where the cytokine or lymphokine is selected from the group consisting of granulocyte macrophage colony stimulating factor and interleukin-12.

64. The adjuvant formulation of claim 61 which further comprises a diluent or carrier.

65. The adjuvant formulation of claim 61 where the cytokine or lymphokine is granulocyte macrophage colony stimulating factor.

66. The adjuvant formulation of claim 61 where the cytokine or lymphokine is interleukin-12.

67. The adjuvant formulation of claim 61 where the AGP is 529.


SEQUENCE LISTING (8 sequences)

SEQ ID NO: 1 - protein, 40 aa, Human immunodeficiency virus
  MISC_FEATURE: Fragment of HIV
KQIINMWQEVGKAMYACTRPNYNKRKRIHIGPGRAFYTTK

SEQ ID NO: 2 - protein, 39 aa, Human immunodeficiency virus
  MISC_FEATURE: Fragment of HIV
KQIINMWQEVGKAMYATRPNYNKRKRIHIGPGRAFYTTK

SEQ ID NO: 3 - protein, 28 aa, Human immunodeficiency virus
  MISC_FEATURE: Fragment of HIV
RQIINTWHKVGKNVYLEGCTPYDINQML

SEQ ID NO: 4 - protein, 39 aa, Human immunodeficiency virus
  MISC_FEATURE: Fragment of HIV
KQIINMWQEVGKAMYATRPNNNTRERLSIGPGRAFYARR

SEQ ID NO: 5 - protein, 39 aa, Human immunodeficiency virus
  MISC_FEATURE: Fragment of HIV
KQIINMWQVVGKAMYATRPNNNTRERLSIGPGRAFYARR

SEQ ID NO: 6 - protein, 42 aa, Homo sapiens [sequence omitted]

SEQ ID NO: 7 - protein, 28 aa, Homo sapiens
  MISC_FEATURE: Fragment of Amyloid Precursor Protein
DAEFRHDSGYEVHHQKLVFFAEDVGSNK

SEQ ID NO: 8 - protein, 10 aa, Human immunodeficiency virus
  MISC_FEATURE: Fragment of HIV
RGPGRAFVTI